(19) United States
(12) Patent Application Publication
(10) Pub. No.: US 2008/0193398 A1
(43) Pub. Date: Aug. 14, 2008

(54) Derivatives of Heptaazaphenalene, Methods for Obtaining Them, and Their Use as Protecting Agents Against Uv Radiation

(75) Inventors: Carles Trullas, Barcelona (ES); Carles Pelejero, Barcelona (ES); David Panyella Costa, Barcelona (ES); Jordi Corbera, Barcelona (ES); Jorg Holenz, Barcelona (ES)

Correspondence Address: BERENBAUM, WEINSHIENK & EASON, P.C, 370 17TH STREET, SUITE 4800, DENVER, CO, 80202, US

(73) Assignee: ISDIN, S.A., Barcelona (ES)

(21) Appl. No.: 11/995,489

(22) PCT Filed: Jul. 13, 2006
(86) PCT No.: PCT/EP2006/064201

§ 371 Date: Jan. 11, 2008

(30) Foreign Application Priority Data
Jul. 13, 2005 (ES) P 200501746

(51) Int. Cl. A61K 8/49; C07D 471/16; A61Q 17/00; A61K 31/53

(52) U.S. Cl. 424/59; 544/209; 514/246

(57) ABSTRACT

The present invention relates to new heptaazaphenalene derivatives of general Formula (I) and to methods for obtaining them. The physicochemical properties of said compounds allow them to be used as UV radiation absorbents.

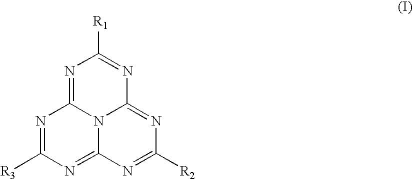

### FIELD OF THE INVENTION

The present invention is related to the cosmetic, dermatological and pharmaceutical fields. In particular, the present invention relates to new derivatives of heptaazaphenalene which, due to their physicochemical properties, are useful as protecting agents against UV radiation, together with their use for manufacturing cosmetic, dermatological, veterinary and pharmaceutical formulations that protect the skin, lips, nails and hair against UV radiation.

### BACKGROUND OF THE INVENTION

Sunlight, and ultraviolet radiation in particular, can under certain circumstances provoke harmful effects on skin, giving rise to pathological manifestations such as sunburns, photodermatosis and photoageing, among others.

The main factor responsible for such pathological manifestations is ultraviolet radiation, whose energy is inversely proportional to its wavelength. Thus, the shorter the wavelength the more energetic the radiation is. Ultraviolet radiation can be classified into UV-C, UV-B and UV-A, with UV-C being the most harmful, although it is absorbed by the ozone layer.

To counteract the damage that UV-A and UV-B radiation can cause, people's skin has various natural protection systems that either absorb or reflect the radiation, such as melanin, hair, the fatty layer of the skin, etc.

Solar filters and/or sunscreens are currently used in this respect in order to reduce the effects of solar radiation. Such UV filters are compounds that are applied to the skin, lips, nails or hair and that can be found included in cosmetic, dermatological and pharmaceutical formulations and in other cosmetic preparations to protect against solar radiation, preventing the decomposition of active substances or components sensitive to radiation.

Research has been carried out in recent years to obtain compounds whose physicochemical properties would be more effective as UV filters.

Despite the wide diversity of solar filters, there exists a need for new compounds whose physicochemical properties make them suitable UV filters to protect against UV-A radiation, UV-B radiation or simultaneously against UV-A and UV-B radiation.

### DESCRIPTION OF THE INVENTION

A first aspect of the invention comprises an heptaazaphenalene derivative of general formula (I):

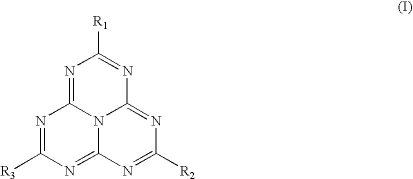

where

R₁, R₂ and R₃ are the same as or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R₅ and R₆ are the same as or different from each other and represent hydrogen; an optionally substituted, saturated or unsaturated, linear or branched radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S;

or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S

or a pharmaceutically, dermatologically or cosmetically acceptable salt, tautomer, isomer or solvate thereof.

In a preferred embodiment, if R₁, R₂ and R₃ are identical they may not be a phenyl, methylphenyl, dimethylphenyl, trimethylphenyl, and 2,3,5,6-tetramethylphenyl.

In another preferred embodiment if R₁, R₂ and R₃ are the same and are OR₄, then R₄ is different from hydrogen.

In another preferred embodiment if R₁, R₂ and R₃ are the same and are OR₄, then R₄ is different from n-butyl, ethyl, phenyl, benzyl, 2,6-dimethylphenyl, 3,5-dimethylphenyl, 2,2,3,3,4,4,4-heptafluorobutyl, 2,2,3,3,3-pentafluoropropyl, 2,2,2-trifluoroethyl and hydroxymethyl.

In another preferred embodiment when R₁, R₂ and R₃ are the same and are OR₄, if R₄ is ethyl for two of the radicals R₁, R₂ and R₃, then R₄ is different from hydrogen for the third R₁, R₂ and R₃ radical.

In another preferred embodiment when R₁, R₂ and R₃ are the same and are NR₅R₆, R₅ and R₆, being the same, are different from hydrogen.

In another preferred embodiment when R₁, R₂ and R₃ are the same and are NR₅R₆, R₅ and R₆, being the same, are different from ethyl, n-butyl, benzyl, n-heptyl, phenyl, cyclohexyl, 2-pyridyl or hydroxymethyl.

In another preferred embodiment when R₁, R₂ and R₃ are the same and are NR₅R₆, if one of R₅ or R₆ is hydrogen, the other radical R₅ or R₆ is different from n-butyl, (optionally unsubstituted) phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl.

In another preferred embodiment when R₁, R₂ and R₃ are the same and are NR₅R₆, if one of R₅ or R₆ is phenyl, the other R₅ or R₆ radical is different from methyl.

In another preferred embodiment when R₁, R₂ and R₃ are NR₅R₆, if R₅ and R₆ are the same for two of the radicals R₁, R₂, R₃, and represent n-heptyl, and for the third radical of R₁, R₂, R₃, being NR₅R₆, R₅ or R₆ is phenyl, then the other R₅ or R₆ is different from phenyl.

In another preferred embodiment when R₁, R₂ and R₃ are NR₅R₆, if R₅ and R₆ are the same for two of the radicals R₁, R₂, R₃, and represent phenyl, and for the third radical of R₁, R₂, R₃, being NR₅R₆, R₅ or R₆ is n-heptyl, then the other R₅ or R₆ is different from n-heptyl.

In a preferred embodiment 6,6′,6″-(1,3,4,6,7,9,9b-heptaazaphenalene-2,5,8-triyltriimino)tris[[(4-acetamido-2-sulfophenyl)azo]-4-hydroxy-2-naphthalenesulfonic acid is disclaimed.
In a preferred embodiment this invention relates to a compound of formula (I) wherein

R₁, R₂ and R₃ are the same as or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; a C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

with the condition that if R₁, R₂ and R₃ are the same, then R₄ is different from hydrogen;

with the condition that if R₁, R₂ and R₃ are the same, then R₄ is different from n-butyl, ethyl, phenyl, benzyl, 2,6-dimethylphenyl, 3,5-dimethylphenyl, 2,2,3,3,4,4,4-heptafluorobutyl, 2,2,3,3,3-pentafluoropropyl, 2,2,2-trifluoroethyl and hydroxymethyl; and

with the condition that if R₄ is ethyl for two of the radicals R₁, R₂ and R₃, then R₄ is different from hydrogen for the third radical;

R₅ and R₆ are the same as or different from each other and represent hydrogen; an optionally substituted, linear or branched radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S;

with the condition that when R₁, R₂ and R₃ are the same, R₅ and R₆, being the same, are different from hydrogen, ethyl, n-butyl, benzyl, n-heptyl, phenyl, cyclohexyl, 2-pyridyl or hydroxymethyl;

with the condition that when R₁, R₂ and R₃ are the same, if R₅ or R₆ is hydrogen, the other radical R₅ or R₆ is different from n-butyl, phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl;

with the condition that when R₁, R₂ and R₃ are the same, if R₅ or R₆ is phenyl, the other R₅ or R₆ radical is different from methyl;

with the condition that if R₅ and R₆ are the same for two of the radicals R₁, R₂, R₃, and represent n-heptyl, and for the third radical of R₁, R₂, R₃, R₅ or R₆ is phenyl, then the other R₅ or R₆ is different from phenyl;

with the condition that if R₅ and R₆ are the same for two of the radicals R₁, R₂, R₃, and represent phenyl, and for the third radical of R₁, R₂, R₃, R₅ or R₆ is n-heptyl, then the other R₅ or R₆ is different from n-heptyl;

or a pharmaceutically, dermatologically or cosmetically acceptable salt, tautomer, isomer or solvate thereof;

In the present invention, “optionally substituted”—if not defined otherwise—means a radical that can be substituted in at least one position, by a linear or branched alkyl radical that contains from 1 to 8 carbon atoms; a C₃-C₆ cycloalkyl radical; C₂-C₆ alkenyl; C₂-C₆ alkenyl-COOR₇; C₂-C₆ alkenyl-aryl; C₁-C₈ alkoxide; aryl; saturated, unsaturated or aromatic heterocyclic group containing from 5 to 10 atoms that can contain 1, 2 or 3 heteroatoms selected from O, N and S; a —COOR₇ radical; a —CONR₈R₉ radical; a —COR₁₀ radical; an hydroxyl radical; an NR₈R₉ radical; a sulfur-containing radical; a nitro radical; an halogen such as chlorine or fluorine; a C₁-C₈ alkoxide; an optionally substituted linear or branched alkyl chain radical having from 1 to 6 carbon atoms, wherein the alkoxide or the alkyl radical can be substituted by at least one hydroxyl group, an —SO₃M radical, a —N(R₁₁)₂ radical, an —N(R₁₁)₃⁺ radical, or a group of general formula (II):

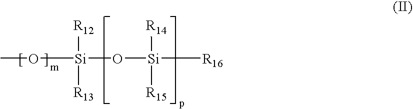

where

m=0 or 1;

p=0, 1, 2, 3 or 4

R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or an —OSi(R₁₇)₃ radical;

R₁₇ represents an alkyl radical having from 1 to 6 carbon atoms, an alkoxide radical having from 1 to 6 carbon atoms or an optionally substituted aryl radical;

M is H, Na or K;

R₇, R₈ and R₉ are independently selected from hydrogen; an optionally substituted linear or branched alkyl radical having from 1 to 18 carbon atoms; a substituted or unsubstituted ary radical; a saturated, unsaturated or aromatic heterocycle having from 5 to 10 atoms that can contain 1, 2 or 3 heteroatoms selected from O, N and S; a C₃-C₁₂ cycloalkyl radical; or

R₈ and R₉ can be fused, forming together with the nitrogen a mono- or polycyclic saturated, unsaturated or aromatic ring system having from 5 to 10 atoms that can optionally contain 1 or 2 heteroatoms selected from O, N and S;

R₁₀ is an optionally substituted alkyl radical, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;

R₁₁ is an optionally substituted alkyl radical.

Any of the above mentioned groups could also be optionally substituted in at least one position.

The term “salt” means any form of the active compound (of general formula (I)) in accordance with the invention in which the latter has an ionic form or it is charged and it is bound to a contra-ion (a cation or an anion) or it is in solution. Also are included complexes of the active compound with other molecules and ions, and in particular complexes that are linked by ionic interactions.

In a preferred meaning the term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes which are complexed via ionic interactions. It especially includes physiologically, dermatologically or cosmetically acceptable salts, which is to be used equivalently to pharmacologically, dermatologically or cosmetically acceptable salts.

The term “pharmaceutically, dermatologically or cosmetically acceptable salt” means, in the context of this invention, the salt formed with a) a pharmaceutically, dermatologically or cosmetically acceptable acid or b) a pharmaceutically acceptable base. This means, especially, salts of the active compound, in particular with inorganic or organic acids that are pharmaceutically, cosmetically and dermatologically acceptable—especially if used on humans and/or mammals—or with at least one cation, preferably inorganic, which is pharmaceutically, cosmetically and dermatologically acceptable—especially if used on humans and/or mammals.

In a preferred meaning the term “pharmaceutically, dermatologically or cosmetically acceptable salt” means, in the context of this invention, the salt formed with a) a pharmaceutically, dermatologically or cosmetically acceptable acid or b) a pharmaceutically, dermatologically or cosmetically acceptable base. This means, especially, salts of the active compound, in particular with inorganic or organic acids that are pharmaceutically, cosmetically and dermatologically acceptable—especially if used on humans and/or mammals—or with at least one cation, preferably inorganic, which is pharmaceutically, cosmetically and dermatologically acceptable—especially if used on humans and/or mammals.

The term “solvate” means, in the context of this invention, a compound formed by the combination of molecules of solvent with molecules or ions of the solute of general formula (I).

In a preferred meaning the term “solvate” according to this invention is to be understood as meaning any form of the active compound according to the invention in which this compound has attached to it via non-covalent binding another molecule (most likely a polar solvent) especially including hydrates and alcoholates, e.g. methanolate.

In a preferred embodiment, the heptaazaphenalene derivative has any of the following general formulas:

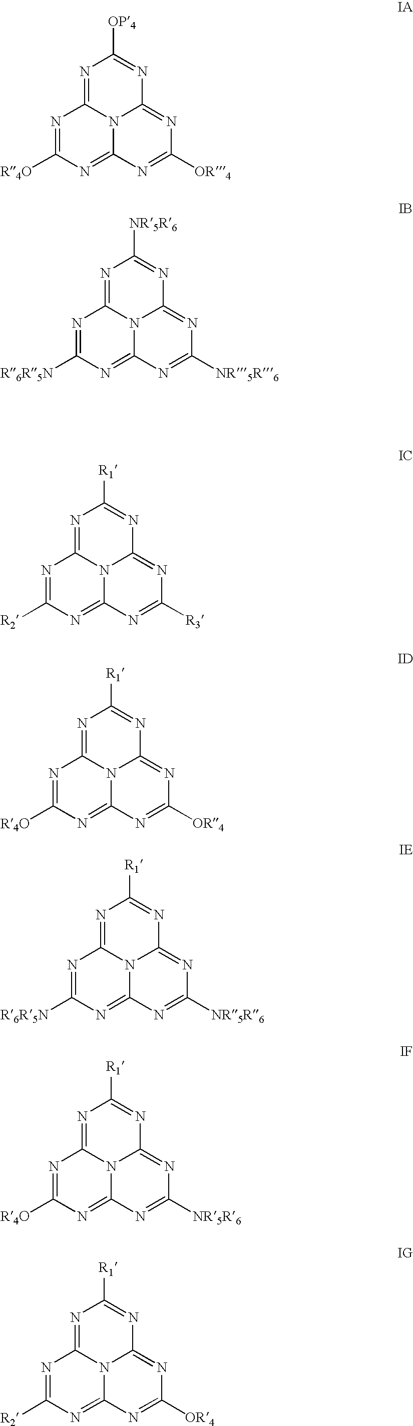

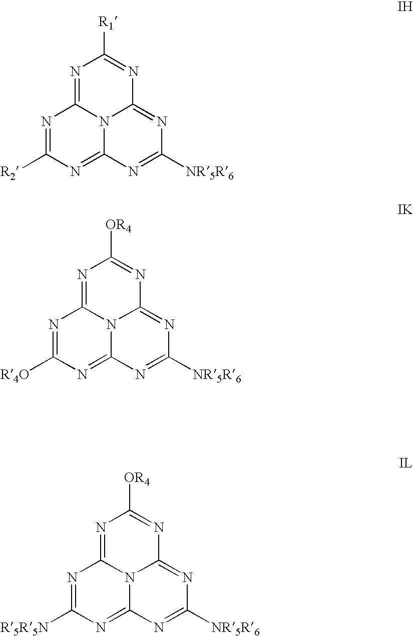

wherein R′₁, R′₂ and R′₃ are the same as or different from each other and represent an optionally substituted mono- or polycyclic aryl radical; an optionally substituted saturated, unsaturated or aromatic heterocyclic radical having from 5 to 10 atoms that can optionally contain 1, 2 or 3 heteroatoms selected from O, N and S;

R′₄, R″₄ and R′″₄ represent independently of each other hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R′₅, R″₅, R′″₅, R′₆, R″₆ and R′″₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R′₅, R″₅, R′″₅, R′₆, R″₆ and R′″₆ are fused to form together with the nitrogen a saturated, unsaturated or aromatic mono- or polycyclic ring system having from 4 to 10 atoms that can optionally contain 1 or 2 heteroatoms selected from N, O and S.

In another preferred embodiment of the first aspect of the invention, the heptaazaphenalene derivative has general formula (IA):

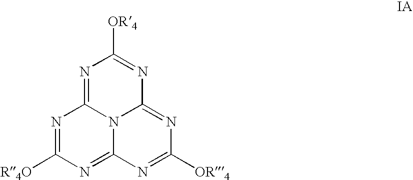

where R′₄, R″₄ and R′″₄ represent independently of each other a cycloalkyl radical having from 3 to 12 carbon atoms; an optionally substituted mono- or polycyclic aryl radical; an optionally substituted saturated, unsaturated or aromatic heterocyclic radical containing from 5 to 14 atoms that can optionally contain 1, 2 or 3 heteroatoms selected from O, N and S.

In another preferred embodiment of the first aspect of the invention, the heptaazaphenalene derivative has general formula (IA):

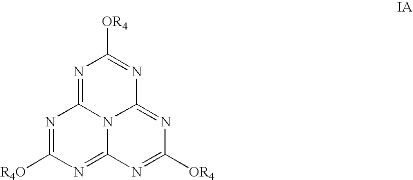

where R₄ represents a cycloalkyl radical having from 3 to 12 carbon atoms; an optionally substituted mono- or polycyclic aryl radical; an optionally substituted saturated, unsaturated or aromatic heterocyclic radical containing from 5 to 14 atoms that can optionally contain 1, 2 or 3 heteroatoms selected from O, N and S.

In a preferred embodiment when R₁, R₂, R₃ are the same, R₄ is different from phenyl, benzyl, 2,6-dimethylphenyl or 3,5-dimethylphenyl.

In a more preferred embodiment, R₄ as well as optionally R′₄, R″₄ and R′″₄ represent an aryl group that can be substituted in at least one position, with said substituent being an optionally substituted C₃-C₁₂ cycloalkyl radical; an optionally substituted C₂-C₆alkenyl radical; an optionally substituted aryl; an optionally substituted saturated, unsaturated or aromatic heterocycle having from 5 to 10 atoms that can contain 1, 2 or 3 heteroatoms selected from O, N and S; a —COOR₇ radical; a —CONR₈R₉ radical; a —COR₁₀ radical; an hydroxyl radical; an halogen such as chlorine or fluorine; C₁-C₈ alkoxide; an optionally substituted linear or branched alkyl chain radical having from 1 to 6 carbon atoms, wherein the alkoxide or the alkyl radical can be substituted by at least one hydroxyl group, an —SO₃M radical, a —N(R₁₁)₂ radical, an —N(R₁₁)₃⁺ radical, or a group of general formula (II):

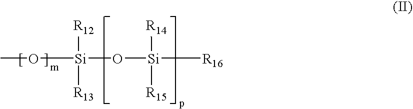

where

m=0 or 1;

p=0, 1, 2, 3 or 4

R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or an —OSi(R₁₇)₃ radical;

R₁₇ represents an alkyl radical having from 1 to 6 carbon atoms, an alkoxide radical having from 1 to 6 carbon atoms or an optionally substituted aryl radical;

M is H, Na or K;

R₇, R₈ and R₉ are independently selected from hydrogen; an optionally substituted or unsubstituted aryl; an optionally substituted linear or branched alkyl radical having from 1 to 18 carbon atoms; a saturated, unsaturated or aromatic heterocycle having from 5 to 10 atoms that can contain 1, 2 or 3 heteroatoms selected from O, N and S; a C₃-C₁₂ cycloalkyl radical; or

R₈ and R₉ can be fused, forming together with the nitrogen a mono- or polycyclic saturated, unsaturated or aromatic ring system having from 5 to 10 atoms that can optionally contain 1 or 2 heteroatoms selected from O, N and S;

R₁₀ is an optionally substituted alkyl radical, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;

R₁₁ is an optionally substituted alkyl radical.

In another preferred embodiment, R₄ represents an aryl group that can be substituted in at least one position, with said substituent being a C₃-C₁₂ cycloalkyl radical; a C₂-C₆ alkenyl; aryl; saturated, unsaturated or aromatic heterocycle having from 5 to 10 atoms that can contain 1, 2 or 3 heteroatoms selected from O, N and S; a —COOR₇ radical; a —CONR₈R₉ radical; a —COR₁₀ radical; an hydroxyl radical; an halogen such as chlorine or fluorine; C₁-C₈ alkoxide; a linear or branched alkyl chain radical having from 1 to 6 carbon atoms, wherein the alkoxide or the alkyl radical can be substituted by at least one hydroxyl group, an —SO₃M radical, a —N(R₁₁)₂ radical, an —N(R₁₁)₃⁺ radical, or a group of general formula (II)

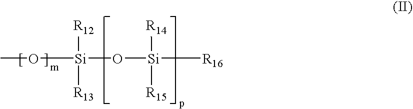

where

m=0 or 1;

p=0, 1, 2, 3 or 4

R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or an —OSi(R₁₇)₃ radical;

R₁₇ represents an alkyl radical having from 1 to 6 carbon atoms, an alkoxide radical having from 1 to 6 carbon atoms or an optionally substituted aryl radical;

M is H, Na or K;

R₇, R₈ and R₉ are independently selected from hydrogen; an optionally substituted linear or branched alkyl radical having from 1 to 18 carbon atoms; a saturated, unsaturated or aromatic heterocycle having from 5 to 10 atoms that can contain 1, 2 or 3 heteroatoms selected from O, N and S; a C₃-C₁₂ cycloalkyl radical; or

R₈ and R₉ can be fused, forming together with the nitrogen a mono- or polycyclic saturated, unsaturated or aromatic ring system having from 5 to 10 atoms that can optionally contain 1 or 2 heteroatoms selected from O, N and S;

R₁₀ is an optionally substituted alkyl radical, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;

R₁₁ is an optionally substituted alkyl radical.

In another preferred embodiment R₄ represents 4-methoxyphenyl, naphthyl, cyclopentyl, cyclohexyl;

with the condition that when R₁, R₂ and R₃ are the same, R₄ is different from phenyl, benzyl, 2,6-dimethylphenyl and 3,5-dimethylphenyl.

In another preferred embodiment of the first aspect of the invention, the heptaazaphenalene derivative has general formula (IB):

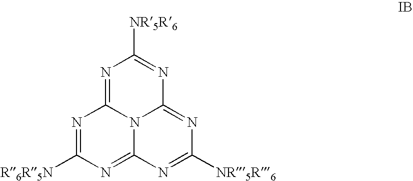

wherein the radicals within each radical pair R′₅R′₆, R″₅R″₆, and R′″₅R′″₆ are different from each other and represent hydrogen; an optionally substituted linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can optionally contain 1, 2 or 3 heteroatoms selected from O, N and S;

or the radical pairs R′₅R′₆, R″₅R″₆, or R′″₅R′″₆ are fused and form together with the nitrogen a saturated, unsaturated or aromatic mono- or polycyclic ring system having from 4 to 10 atoms that can optionally contain 1 or 2 heteroatoms selected from N, O and S.

In another preferred embodiment of the first aspect of the invention, the heptaazaphenalene derivative has general formula (IB):

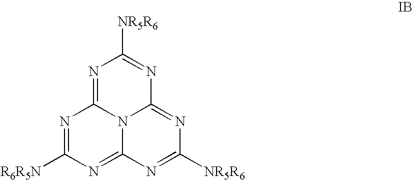

R₅ and R₆ are different from each other and represent hydrogen; an optionally substituted linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can optionally contain 1, 2 or 3 heteroatoms selected from O, N and S;

or R₅ and R₆ are fused and form together with the nitrogen a saturated, unsaturated or aromatic mono- or polycyclic ring system having from 4 to 10 atoms that can optionally contain 1 or 2 heteroatoms selected from N, O and S.

In a preferred embodiment if one of the radicals within each radical pair R′₅R′₆, R″₅R″₆, and R′″₅R′″₆ is hydrogen, the other radical is different from n-butyl, unsubstituted phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl.

In another preferred embodiment if one of the radicals within each radical pair R′₅R′₆, R″₅R″₆, and R′″₅R′″₆ is phenyl, the other radical is different from methyl.

In a more preferred embodiment one radical of the pair R₅R₆ or optionally one radical of the pairs R′₅R′₆, R″₅R″₆ or R′″₅R′″₆ represents an aryl group that can be substituted in at least one position, with said substituent being a C₃-C₁₂ cycloalkyl radical; an optionally substituted C₂-C₆ alkenyl radical; an optionally substituted aryl radical; an optionally substituted saturated, unsaturated or aromatic heterocycle having from 5 to 10 atoms that can contain 1, 2 or 3 heteroatoms selected from O, N and S; a —COOR₇ radical; a —CONR₈R₉ radical; a —COR₁₀ radical; an hydroxyl radical; an halogen such as chlorine or fluorine; C₁-C₈ alkoxide; an optionally substituted linear or branched alkyl radical having from 1 to 6 carbon atoms, where the alkoxide radical or the alkyl radical can be optionally substituted by at least one —SO₃M group, an —N(R₁₁)₂ radical, an —N(R₁₁)₃⁺ radical, or a group of general formula (II):

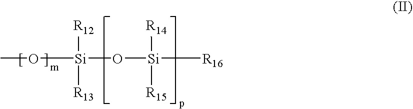

where

m=0 or 1;

p=0, 1, 2, 3 or 4

R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or a —OSi(R₁₇)₃ radical;

R₁₇ represents an alkyl radical having from 1 to 6 carbon atoms, an alkoxide radical having from 1 to 6 carbon atoms or an optionally substituted aryl radical;

M is H, Na or K;

R₇, R₈ and R₉ are independently selected from hydrogen; an optionally substituted aryl radical; an optionally substituted linear or branched alkyl radical having from 1 to 18 carbon atoms; an optionally substituted saturated, unsaturated or aromatic heterocycle having from 5 to 10 atoms that can contain 1, 2 or 3 heteroatoms selected from O, N and S; a C₃-C₁₂ cycloalkyl radical; or

R₈ and R₉ can be fused to form together with the nitrogen a mono- or polycyclic saturated, unsaturated or aromatic ring system having from 5 to 10 atoms that can optionally contain 1 or 2 heteroatoms selected from O, N and S;

R₁₀ is an optionally substituted saturated or unsaturated, linear or branched alkyl radical having from 1 to 6 carbon atoms, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;

R₁₁ is an optionally substituted alkyl radical.

In another preferred embodiment R₅ or R₆ represent an aryl group that can be substituted in at least one position, with said substituent being a C₃-C₁₂ cycloalkyl radical; a C₂-C₆alkenyl; an aryl; a saturated, unsaturated or aromatic heterocycle having from 5 to 10 atoms that can contain 1, 2 or 3 heteroatoms selected from O, N and S; a —COOR₇ radical; a —CONR₈R₉ radical; a —COR₁₀ radical; an hydroxyl radical; an halogen such as chlorine or fluorine; C₁-C₈ alkoxide; a linear or branched alkyl radical having from 1 to 6 carbon atoms, where the alkoxide radical or the alkyl radical can be substituted by —SO₃M group, an —N(R₁₁)₂ radical, an —N(R₁₁)₃⁺ radical, or a group of general formula (II):

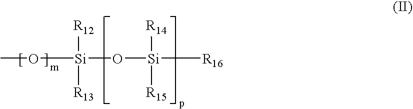

where

m=0 or 1;

p=0, 1, 2, 3 or 4

R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or a —OSi(R₁₇)₃ radical;

R₁₇ represents an alkyl radical having from 1 to 6 carbon atoms, an alkoxide radical having from 1 to 6 carbon atoms or an optionally substituted aryl radical;

M is H, Na or K;

R₇, R₈ and R₉ are independently selected from hydrogen; an optionally substituted linear or branched alkyl radical having from 1 to 18 carbon atoms; a saturated, unsaturated or aromatic heterocycle having from 5 to 10 atoms that can contain 1, 2 or 3 heteroatoms selected from O, N and S; a C₃-C₁₂ cycloalkyl radical; or

R₈ and R₉ can be fused to form together with the nitrogen a mono- or polycyclic saturated, unsaturated or aromatic ring system having from 5 to 10 atoms that can optionally contain 1 or 2 heteroatoms selected from O, N and S;

R₁₀ is an optionally substituted saturated or unsaturated, linear or branched alkyl radical having from 1 to 6 carbon atoms, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;

R₁₁ is an optionally substituted alkyl radical.

In another preferred embodiment, the heptaazaphenalene derivative has general formula (IC)

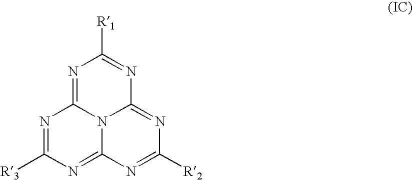

wherein R′₁, R′₂ and R′₃ are the same as or different from each other and represent an optionally substituted mono- or polycyclic aryl radical; an optionally substituted saturated, unsaturated or aromatic heterocyclic radical having from 5 to 10 atoms that can optionally contain 1, 2 or 3 heteroatoms selected from O, N and S.

In a preferred embodiment, if R′₁, R′₂ and R′₃ are identical they may not be a phenyl, methylphenyl, dimethylphenyl, trimethylphenyl, and 2,3,5,6-tetramethylphenyl.

In a yet more preferred embodiment R₁, R₂, R₃ as well as optionally R′₁, R′₂, R′₃ are the same as or different from each other and represent naphthyl, pyrrole, thiophene, indole, pyrazole, imidazole, triazole, benzothiophene, benzimidazole, benzopyrazole, oxazole, isoxazole, benzofuran, all of them optionally substituted, or else a radical of general formula (III)

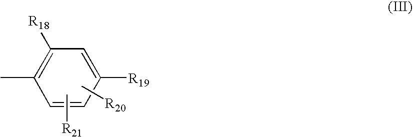

R₁₈ represents an hydrogen; or an hydroxyl radical; an —OR₂₂ radical; an optionally substituted saturated or unsaturated linear or branched alkyl radical that contains from 1 to 18 carbon atoms; or an optionally substituted linear or branched chain C₁-C₁₈ alkoxyde radical;

R₁₉ represents an hydrogen; an hydroxyl radical; an optionally substituted aryl radical; an optionally substituted saturated, unsaturated or aromatic heterocyclic radical having from 5 to 10 atoms that can optionally contain 1, 2 or 3 heteroatoms selected from O, N and S; a —COOR₇ radical; a —CONR₈R₉ radical; an —OR₂₂ radical; an optionally substituted —COR₁₀ radical; a C₃-C₆ cycloalkyl radical; an optionally substituted, saturated or unsaturated linear or branched alkyl radical that contains from 1 to 18 carbon atoms, optionally substituted by at least one hydroxyl radical, an —SO₃M, —N(R₁₁)₂ or —N(R₁₁)₃⁺ group, or else by a group of general formula (II):

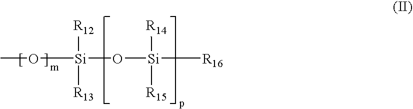

wherein

m=0 or 1;

p=0, 1, 2, 3 or 4;

R₂₂ represents an optionally substituted aryl radical; an optionally substituted saturated, unsaturated or aromatic heterocyclic radical having from 5 to 10 atoms that can optionally contain 1, 2 or 3 heteroatoms selected from O, N and S; an optionally substituted —COR₁₀ radical; a C₃-C₁₂ cycloalkyl radical; a saturated or unsaturated linear or branched alkyl radical that contains from 1 to 18 carbon atoms, optionally substituted at least by one hydroxyl radical, a —SO₃M, —N(R₁₁)₂ or —N(R₁₁)₃⁺ group or else by a group of general formula (II):

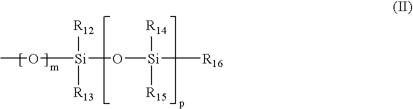

wherein

m=0 or 1;

p=0, 1, 2, 3 or 4;

where R₇, R₈, R₉, R₁₀, R₁₁, R₁₂, R₁₃, R₁₄, R₁₅ and R₁₆ are as defined above;

R₂₀ and R₂₁ can be the same or different and represent hydrogen; an optionally substituted saturated or unsaturated linear or branched alkyl radical that contains from 1 to 6 carbon atoms; an optionally substituted C₁-C₆ alkoxide radical; or an —SO₃M radical, where M is as defined above.

In another more preferred embodiment R₅ and R₆ are different from each other and represent hydrogen, cyclopropyl, cyclohexyl, cyclobutyl, cycloheptyl, cyclopentyl, 4-(hydroxycarbonyl)phenyl, 4-(butoxycarbonyl)phenyl, 4-(2-ethylhexyloxycarbonyl)phenyl, 4-(2-butyloctyloxycarbonyl)phenyl, 4-(2-hexyldecyloxycarbonyl)phenyl, 4-(3,3,5-trimethylcyclohexyloxycarbonyl)phenyl, 4-(3,3,5-trimethylhexyloxycarbonyl)phenyl, 4-(octadecyloxycarbonyl)phenyl, 4-(hexadecyloxycarbonyl)phenyl, 4-(docecyloxycarbonyl)phenyl, 4-((2-ethylhexyl)carbamoyl)phenyl, 4-(L-menthyloxycarbonyl)phenyl, 4-styrylphenyl, 3-styrylphenyl, 3-((E)-3-(2-ethylhexyloxy)-3-oxoprop-1-enyl)phenyl, 4-((E)-3-(2-ethylhexyloxy)-3-oxoprop-1-enyl)phenyl, 4-methoxyphenyl, 3-methoxyphenyl, 3-nitrophenyl phenyl, biphenyl-4-yl, 4-(imidazo[1,2-a]pyridin-2-yl)phenyl, 4-(4,5,6,7-tetrahydro-2,6,6-trimethyl-4-oxoindol-1-yl)phenyl, 4-(1H-benzo[d]imidazol-2-yl)phenyl, hydroxymethyl, pyridine, heptyl, butyl, ethyl, 2-ethylhexyl, 4-(1,3,3-trimethylbicyclo[2.2.1]heptan-2-yloxy)phenyl, 1H-indol-5-yl, 4-((3,7-dimethyloctyloxy)carbonyl)phenyl, 2-amino-4,5-dimethylphenyl or n-propyl.

In another more preferred embodiment R₄ represents 4-methoxyphenyl, naphthyl, cyclopentyl, cyclohexyl.

In still another more preferred embodiment when R₁, R₂ and R₃ are the same, R₄ is different from phenyl, benzyl, 2,6-dimethylphenyl and 3,5-dimethylphenyl.

In another preferred embodiment R₇ represents hydrogen, methyl, ethyl, n-propyl, isopropyl, n-butyl, tert-butyl, n-pentyl, n-hexyl, 2-ethylhexyl, L-menthyl, 3,3,5-trimethylcyclohexanyl, 3,3,5-trimethylhexanyl, dodecyl, 2-butyloctyl, 2-hexyldecyl, octadecyl, 3,7-dimethyloctyl, 1,3,3-trimethylbicyclo[2.2.1]heptan-2-yl, optionally substituted benzyl radical or an optionally substituted phenyl radical.

In another preferred embodiment R₈ and R₉, are independently selected from hydrogen, methyl, ethyl, n-propyl, isopropyl, n-butyl, tert-butyl, n-pentyl, n-hexyl 2-ethylhexyl, L-menthyl, 3,3,5-trimethylcyclohexanyl, 3,3,5-trimethylhexanyl, dodecyl, 2-butyloctyl, 2-hexyldecyl, 3,7-dimethyloctyl, 1,3,3-trimethylbicyclo[2.2.1]heptan-2-yl or octadecyl.

In another preferred embodiment of the first aspect of the invention R₁₀ represents methyl, ethyl, n-propyl, n-butyl, tert-butyl or phenyl.

In another preferred embodiment R₁₂ to R₁₆ represent methyl, ethyl, methoxy, ethoxy or phenyl.

In another preferred embodiment R₁₇ represents methyl, ethyl, methoxy, ethoxy or phenyl.

In another preferred embodiment of the first aspect of the invention, R₁₈ represents hydrogen, an hydroxyl radical, a methyl radical, a methoxy radical or an acyloxy radical.

In another preferred embodiment R₁₉ represents an hydrogen, a hydroxyl radical, an acyloxy radical, a linear or branched chain, saturated or unsaturated alkoxide radical such as methoxy, ethoxy, n-propoxy, iso-propoxy, n-butoxy, tert-butoxy, 2-ethylhexyloxy, phenoxide, optionally substituted by at least one —SO₃M or —N(R₁₁)₃⁺ group.

In another preferred embodiment of the first aspect of the invention R₂₀ and R₂₁ are independently selected from hydrogen, a hydroxyl radical, methoxy, ethoxy, n-propoxy, iso-propoxy, n-butoxy, tert-butoxy, n-pentoxy, hexyloxy or 2-ethylhexyloxy, optionally substituted by at least one —SO₃M group, where M is as defined above.

Preferably the heptaazaphenalene derivative of general formula (I) is selected from the group that consists in:
2,5,8-tris-(4-(butoxycarbonyl)phenylamino)-1,3,4,6,7,9,9b-heptaazaphenalene;
2,5,8-tris-(4-(2-ethylhexyloxycarbonyl)phenylamino)-1,3,4,6,7,9,9b-heptaazaphenalene;
2,5,8-tris-(4-(imidazo[1,2-a]pyrridin-2-yl)-phenylamino)-1,3,4,6,7,9,9b-heptaazaphenalene;
2,5,8-tris-(4-(4,5,6,7-tetrahydro-2,6,6-trimethyl-4-oxoindol-1-yl)phenylamine)-1,3,4,6,7,9,9b-heptaazaphenalene;
2,5,8-tris-(biphenyl-4-ylamino)-1,3,4,6,7,9,9b-heptaazaphenalene;
2,5,8-tris-(4-(1H-benzo[d]imidazol-2-yl)phenylamino)-1,3,4,6,7,9,9b-heptaazaphenalene;
2,5-bis-(biphenyl-4-ylamino)-8-(4-(butoxycarbonyl)phenylamino)-1,3,4,6,7,9,9b-heptaazaphenalene;
2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene;
2,5,8-tris-(2,4-dihydroxyphenyl)-1,3,4,6,7,9,9b-heptaazaphenalene;
2,5-dichloro-8-(1-methyl-1H-pyrrol-2-yl)-1,3,4,6,7,9,9b-heptaazaphenalene;
2,5-bis-(4-(butoxycarbonyl)phenylamino)-8-(2-(1-methyl-1H-pyrrol-2-yl)-1,3,4,6,7,9, 9b-heptaazaphenalene;
2-(4-(carboxy)phenylamino)-5,8-bis-(4-methylphenyl)-1,3,4,6,7,9,9b-heptaazaphenalene;
2,5,8-tris-(4-(4,5,6,7-tetrahydro-2,6,6-trimethyl-4-oxoindol-1-yl)phenylamine)-1,3,4,6,7,9, 9b-heptaazaphenalene;
2,5-bis-(4-(butoxycarbonyl)phenylamino)-8-(1-methyl-1H-pyrrol-2-yl)-1,3,4,6,7,9,9b-heptaazaphenalene;
2,5-bis(2,4-dihydroxyphenyl)-8-(1-methyl-1H-pyrazol-5-yl)-1,3,4,6,7,9,9b-heptaazaphenalene;
2,5-bis{[4-(2-ethylhexyloxy)-2-hydroxy]phenyl}-6-(1-methyl-1H-pyrazol-5-yl)-1,3,4,6,7,9,9b-heptaazaphenalene;
2-[2,4-bis(2-ethylhexyloxy)phenyl]-5-[4-(2-ethylhexyloxy)-2-hydroxyphenyl]-8-(1-methyl-1H-pyrazol-5-yl)-1,3,4,6,7,9,9b-heptaazaphenalene;
2,5-bis(2,4-dihydroxyphenyl)-8-(1-phenyl-1H-pyrazol-5-yl)-1,3,4,6,7,9,9b-heptaazaphenalene;
2,5-bis{[4-(2-ethylhexyloxy)-2-hydroxy]phenyl}-8-(1-phenyl-1H-pyrazol-5-yl)-1,3,4,6,7,9,9b-heptaazaphenalene;
2,5-bis[4-(butoxycarbonyl)phenylamino]-8-(1-methyl-1H-pyrazol-5-yl)-1,3,4,6,7,9, 9b-heptaazaphenalene;
2,5-bis{4-[(2-(ethylhexyloxy)carbonyl]phenylamino}-8-(1-methyl-1H-pyrazol-5-yl)-1,3,4,6,7,9,9b-heptaazaphenalene;
2,5-bis[4-(butoxycarbonyl)phenylamino]-8-(1-phenyl-1H-pyrazol-5-yl)-1,3,4,6,7,9,9b-heptaazaphenalene;
2,5-bis{4-[(2-ethylhexyloxy)carbonyl]phenylamino}-8-(1-phenyl-1H-pyrazol-5-yl)-1,3,4,6,7,9,9b-heptaazaphenalene;
2-(1-benzyl-1H-pyrrol-2-yl)-5,8-bis(2,4-dihydroxyphenyl)-1,3,4,6,7,9,9b-heptaazaphenalene;
2-(1-benzyl-1H-pyrrol-2-yl)-5,8-bis[4-(2-ethylhexyloxy)-2-hydroxyphenyl]-1,3,4,6,7,9,9b-heptaazaphenalene;
2-(1-benzyl-1H-pyrrol-2-yl)-5,8-bis[4-(butoxycarbonyl)phenylamino]-1,3,4,6,7,9,9b-heptaazaphenalene;
2-(1-benzyl-1H-pyrrol-2-yl)-5,8-bis(biphenyl-4-ylamino)-1,3,4,6,7,9,9b-heptaazaphenalene;
2-(1-benzyl-1H-pyrrol-2-yl)-5,8-bis(4-benzoylphenylamino)-1,3,4,6,7,9,9b-heptaazaphenalene;
2-(1-benzyl-1H-pyrrol-2-yl)-5,8-bis(9-oxo-9H-fluoren-3-ylamino)-1,3,4,6,7,9,9b-heptaazaphenalene;
2-(4-(tert-butylcarbamoyl)phenylamino)-5,8-bis-(4-(2-ethylhexyloxycarbonyl)phenylamino)-1,3,4,6,7,9,9b-heptaazaphenalene;
2,5-bis-[(4-(2-ethylhexyloxy)-2-hydroxy)-phenyl]-8-(4-methoxyphenyl)-1,3,4,6,7,9,9b-heptaazaphenalene;
2-(2-ethylhexylamino)-5,8-bis-(4-(5-(1,1-dimethylpropyl)benzo[d]oxazol-2-yl)phenylamino)-1,3,4,6,7,9,9b-heptaazaphenalene;
2,5,8-tris-(4-(1H-pyrazol-1-yl)phenylamino)-1,3,4,6,7,9,9b-heptaazaphenalene
2,5,8-tris-(4-benzoylphenylamino)-1,3,4,6,7,9,9b-heptaazaphenalene.
2,5,8-tris-(4-butoxy-2-hydroxyphenyl)-1,3,4,6,7,9,9b-heptaazaphenalene.
2,5,8-tris-(naphthalen-2-ylamino)-1,3,4,6,7,9,9b-heptaazaphenalene.
2,5-bis-(4-(butoxycarbonyl)phenylamino)-8-(2-(1-methyl-1H-indol-3-yl)-1,3,4,6,7,9,9b-heptaazaphenalene.
2,5,8-tris-(biphenyl-4-yloxy)-1,3,4,6,7,9,9b-heptaazaphenalene.
2,5,8-tris-(3-methoxyphenylamino)-1,3,4,6,7,9,9b-heptaazaphenalene.
2,5,8-tris-(4-methoxyphenylamino)-1,3,4,6,7,9,9b-heptaazaphenalene.
2,5,8-tris-(4-((E)-3-ethoxy-3-oxoprop-1-enyl)phenylamino)-1,3,4,6,7,9,9b-heptaazaphenalene.
2,5,8-tris-(methoxycarbonyl-4′-biphenyl-4-ylamino)-1,3,4,6,7,9,9b-heptaazaphenalene.
2,5,8-tris-(4-(methoxycarbonyl)phenylamino)-1,3,4,6,7,9,9b-heptaazaphenalene.
2,5,8-tris-(4-(1H-benzo[d]imidazol-2-yl)-3-hydroxyphenylamino)-1,3,4,6,7,9,9b-heptaazaphenalene.
2,5,8-tris-(4-(phenylamino)phenylamino)-1,3,4,6,7,9,9b-heptaazaphenalene.
2,5,8-tris-((4-(E)-styrylphenyl)amino)-1,3,4,6,7,9,9b-heptaazaphenalene.
2,5,8-tris-(2-ethylhexylamino)-1,3,4,6,7,9,9b-heptaazaphenalene.
2,5,8-tris-(4-(L-menthylcarbonyl)phenylamino)-1,3,4,6,7,9,9b-heptaazaphenalene.
2,5,8-tris-(4-((3,3,5-trimethylcyclohexyloxy)carbonyl)phenylamino)-1,3,4,6,7,9,9b-heptaazaphenalene.
2,5,8-tris-(4-((E)-3-(2-ethylhexyloxy)-3-oxoprop-1-enyl)phenylamino)-1,3,4,6,7,9,9b-heptaazaphenalene.
2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene.
2,5,8-tris-(3-nitrophenylamino)-1,3,4,6,7,9,9b-heptaazaphenalene.
2,5,8-tris-(4-(2-butyloctyloxycarbonyl)phenylamino)-1,3,4,6,7,9,9b-heptaazaphenalene.
2,5,8-tris-(4-(2-hexyldecyloxycarbonyl)phenylamino)-1,3,4,6,7,9,9b-heptaazaphenalene.
2,5,8-tris-(4-(dodecyloxycarbonyl)phenylamino)-1,3,4,6,7,9,9b-heptaazaphenalene.
2,5,8-tris-(4-(hexyldecyloxycarbonyl)phenylamino)-1,3,4,6,7,9,9b-heptaazaphenalene.
2,5,8-tris-(4-(octyldecyloxycarbonyl)phenylamino)-1,3,4,6,7,9,9b-heptaazaphenalene.
2,5,8-tris-((3-(E)-styrylphenyl)amino)-1,3,4,6,7,9,9b-heptaazaphenalene.
2,5,8-tris-(4-((3,5,5-trimethylhexyloxy)carbonyl)phenylamino)-1,3,4,6,7,9,9b-heptaazaphenalene.
2,5-bis-(3-(methoxy)phenylamino)-8-(2-(1-methyl-1H-pyrrol-2-yl)-1,3,4,6,7,9,9b-heptaazaphenalene.
2,5,8-tris-(4-((2-ethylhexyl)carbamoyl)phenylamino)-1,3,4,6,7,9,9b-heptaazaphenalene.
2,5,8-tris-(4-(dodecyloxycarbonyl)phenylamino)-1,3,4,6,7,9,9b-heptaazaphenalene.
2,5,8-tris-(4-(1,3,3-trimethylbicyclo[2.2.1]heptan-2-yloxy)phenylamino)-1,3,4,6,7,9,9b-heptaazaphenalene.
2,5,8-tris-(1H-indol-5-ylamino)-1,3,4,6,7,9,9b-heptaazaphenalene.
2,5,8-tris-(4-((3,7-dimethyloctyloxy)carbonyl)phenylamino)-1,3,4,6,7,9,9b-heptaazaphenalene.
2,5,8-tris-(2-amino-4,5-dimethylphenylamino)-1,3,4,6,7,9,9b-heptaazaphenalene.
   Other possible examples for compounds of general formula (I) are shown in Table (I):

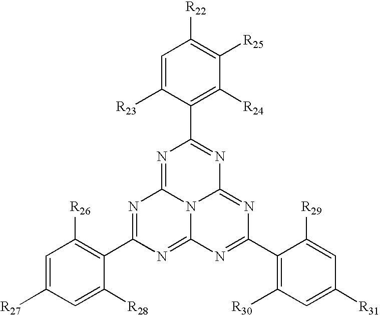

| R₂₂ | R₂₃ | R₂₄ | R₂₅ | R₂₆ | R₂₇ |
|---|---|---|---|---|---|
| —OMe | H | H | H | —OH | 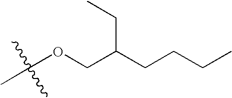 |
| 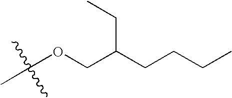 | OH | H | H | Me | Me |
| 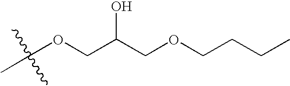 | OH | H | H | Me | Me |
| 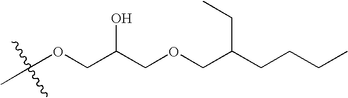 | OH | H | H | Me | Me |
| 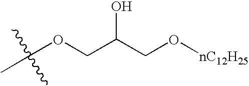 | OH | H | H | Me | Me |
| 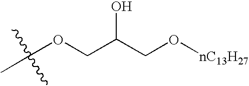 | OH | H | H | Me | Me |
| 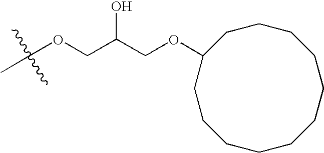 | OH | H | H | Me | Me |
| 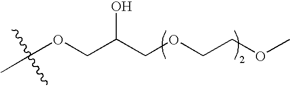 | OH | H | H | Me | Me |
| 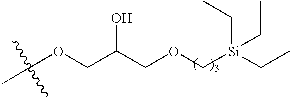 | OH | H | H | Me | Me |
| 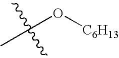 | H | H | H | H | H |
| —OAc | OAc | H | H | H | H |
| 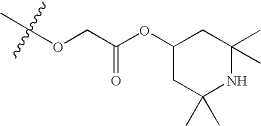 | H | H | H | Me | Me |
| 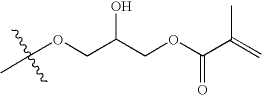 | H | H | H | Me | Me |
| —OMe | OH | H | H | H | Ph |
| —OBu | OH | H | H | H | Ph |
| 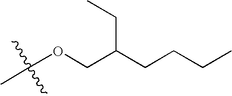 | OH | H | H | H | Ph |
| 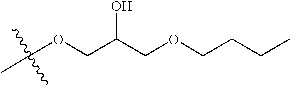 | OH | H | H | H | Ph |
| 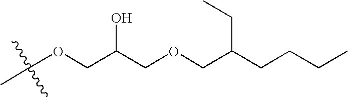 | OH | H | H | H | Ph |
| 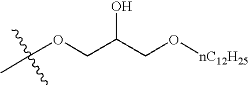 | OH | H | H | H | Ph |
| 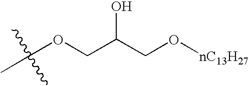 | OH | H | H | H | Ph |
| 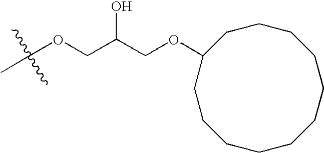 | OH | H | H | H | Ph |
| 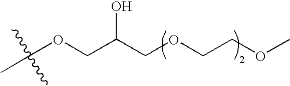 | OH | H | H | H | Ph |
| 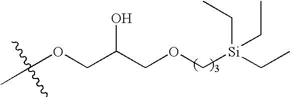 | OH | H | H | H | Ph |
| 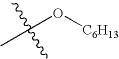 | H | H | H | H | Ph |
| 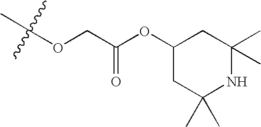 | H | H | H | H | Ph |
| 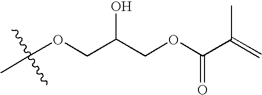 | H | H | H | H | Ph |
| H | H | H | H | H | H |
| H | OMe | H | H | H | H |
| —OMe | H | H | H | H | H |
| —OMe | OH | H | H | H | H |
| —OMe | OH | H | H | H | Me |
| —OMe | OH | H | H | Me | Me |
| —OMe | OH | H | H | OMe | —OMe |
| —OC₆H₁₃ | OH | H | H | Me | Me |
| —OC₆H₁₃ | OMe | H | H | H | H |
| —OC₆H₁₃ | OH | H | H | H | —OMe |
| —OC₆H₁₃ | OH | H | H | Me | Me |
| —OC₆H₁₃ | OMe | H | H | Me | Me |
| —OC₆H₁₃ | OH | H | Me | H | H |
| —OC₆H₁₃ | OH | Me | H | H | H |
| 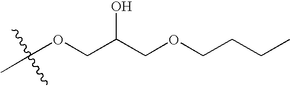 | OH | H | H | —OH | 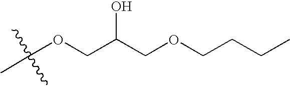 |
| 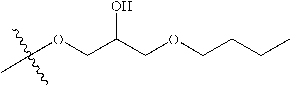 | OH | H | H | —OH | 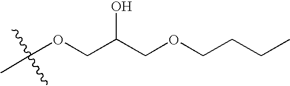 |
| 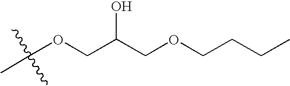 | OH | H | H | —OH | 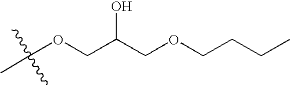 |
| —OMe | OH | H | H | —OH | 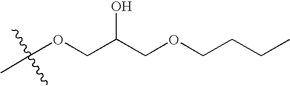 |
| 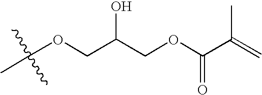 | OH | H | H | Me | Me |
| —OC₁₂H₂₅ | OH | H | H | Me | Me |
| —OMe | OMe | H | H | H | Me |
| —OMe | OMe | H | H | Me | Me |
| —OC₁₂H₂₅ | OMe | H | H | H | H |
| —OC₁₂H₂₅ | OMe | H | H | H | Me |
| —OC₁₂H₂₅ | OMe | H | H | Me | Me |
| H | H | H | H | —OH | 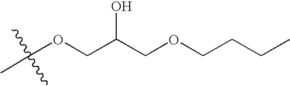 |
| —OMe | OMe | H | H | H | H |
| —OC₁₂H₂₅ | Ome | H | H | H | H |
| —OMe | Ome | H | H | H | Me |
| —OC₆H₁₃ | Ome | H | H | H | Me |
| —OMe | Ome | H | H | Me | Me |
| —OC₁₂H₂₅ | Ome | H | H | Me | Me |
| H | Ome | H | H | H | H |
| —OMe | H | H | H | H | H |
| —OMe | H | H | H | H | —OMe |
| —OMe | H | H | H | H | —OMe |
| H | —OH | H | H | H | H |
| H | —OH | H | H | —OH | H |
| H | H | H | H | H | H |
| 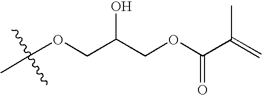 | —OH | H | H | Me | Me |
| —OMe | —OH | H | H | —OMe | —OMe |
| —OMe | —OH | H | H | —OH | —OMe |
| —OMe | —OH | H | H | H | H |
| 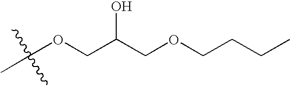 | —OH | H | H | —OH | 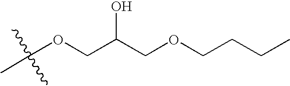 |

| R₂₈ | R₂₉ | R₃₀ | R₃₁ |
|---|---|---|---|
| H | —OH | H | 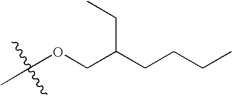 |
| H | Me | H | Me |
| H | Me | H | Me |
| H | Me | H | Me |
| H | Me | H | Me |
| H | Me | H | Me |
| H | Me | H | Me |
| H | Me | H | Me |
| H | Me | H | Me |
| H | H | H | H |
| H | H | H | H |
| H | Me | H | Me |
| H | Me | H | Me |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | Ph |
| H | H | H | H |
| H | H | H | H |
| H | H | H | H |
| H | H | H | H |
| H | H | H | Me |
| H | Me | H | Me |
| H | OMe | H | —OMe |
| H | Me | H | Me |
| H | H | H | H |
| H | H | H | —OMe |
| Me | Me | Me | Me |
| Me | Me | Me | Me |
| H | H | H | H |
| H | H | H | H |
| H | H | H | H |
| H | Me | H | Me |
| H | Me | Me | Me |
| H | —OH | H | 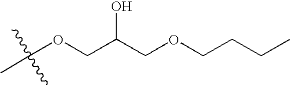 |
| H | Me | H | Me |
| H | Me | H | Me |
| H | H | H | Me |
| H | Me | H | Me |
| H | H | H | H |
| H | H | H | Me |
| H | Me | H | Me |
| H | —OH | H | 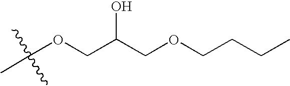 |
| H | H | H | H |
| H | H | H | H |
| H | H | H | Me |
| H | H | H | Me |
| H | H | H | Me |
| H | Me | H | Me |
| H | Me | H | H |
| H | H | H | H |
| H | H | H | H |
| H | H | H | —OMe |
| H | H | H | H |
| H | H | H | H |
| H | H | H | H |
| H | Me | H | Me |
| H | OMe | H | —OMe |
| H | OMe | H | —OMe |
| H | H | H | H |
| H | H | H | H |

Other possible examples for compounds of general formula (I) are shown in Table (II):

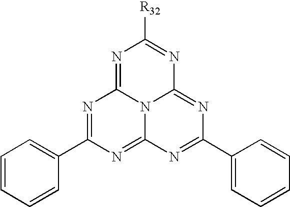

R₃₂

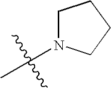

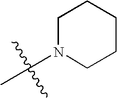

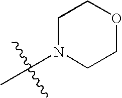

Other possible examples for compounds of general formula (I) are shown in Table (III):

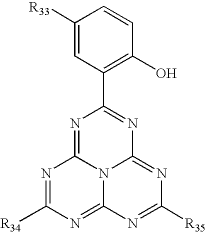

| R₃₃ | R₃₄ | R₃₅ |
|---|---|---|
| H | —OMe | —OMe |
| Me | —OMe | —OMe |
| Me | —OMe | —N(Me)₂ |
| Me | —N(Me)₂ | —N(Me)₂ |

Other possible examples for compounds of general formula (I) are shown in Table (IV):

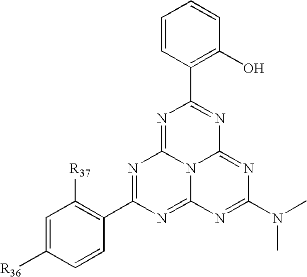

| R₃₆ | R₃₇ |
|---|---|
| H | H |
| H | —OH |
| Me | H |

Other possible examples for compounds of general formula (I) are shown in Table (V):

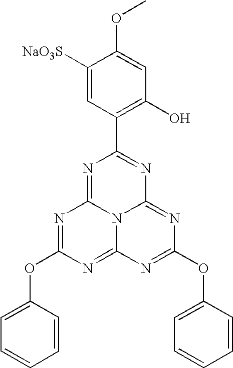

Other possible examples for compounds of general formula (I) are shown in Table (VI):

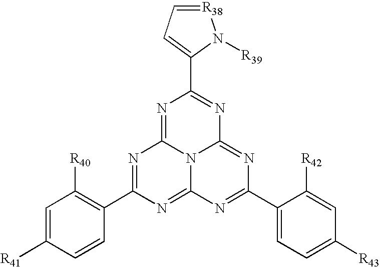

| R₃₈ | R₃₉ | R₄₀ | R₄₁ | R₄₂ | R₄₃ |
|---|---|---|---|---|---|
| N | Me | —OH | —OH | —OH | —OH |
| N | Me | —OH | 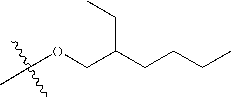 | —OH | 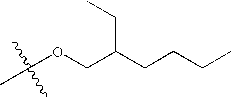 |
| N | Me | 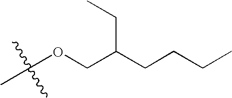 | 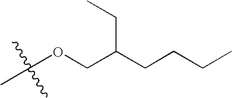 | —OH | 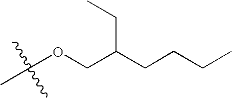 |
| N | Ph | —OH | —OH | —OH | —OH |
| N | Ph | —OH | 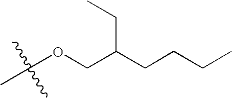 | —OH | 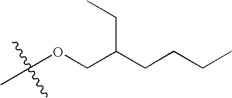 |
| —CH | CH₂Ph | —OH | —OH | —OH | —OH |
| —CH | CH₂Ph | —OH | 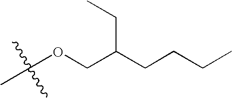 | —OH | 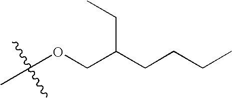 |

Other possible examples for compounds of general formula (I) are shown in Table (VII):

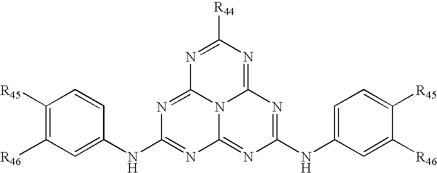

| R₄₄ | R₄₅ | R₄₆ |
|---|---|---|
| 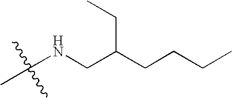 | 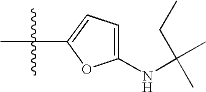 | H |
| 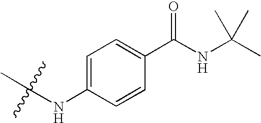 | 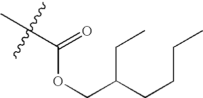 | H |
| 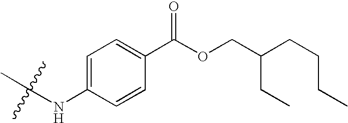 | 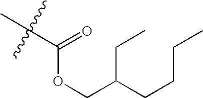 | H |
| 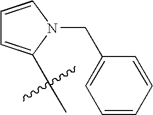 | Ph | H |
| 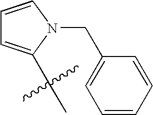 | 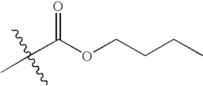 | H |
| 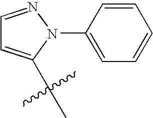 | 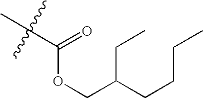 | H |
| 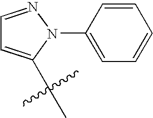 | 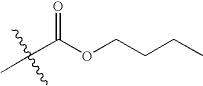 | H |
| 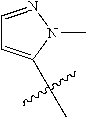 | 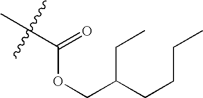 | H |
| 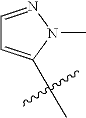 | 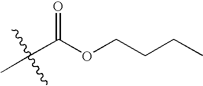 | H |
| 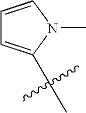 | 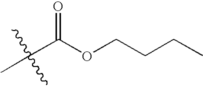 | H |
| 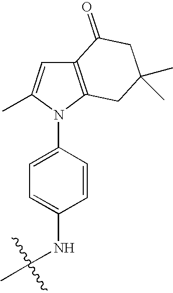 | 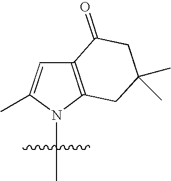 | H |
| 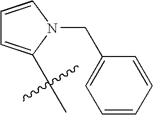 | —COPh | H |

Surprisingly, the inventors of the present invention have found that the heptaazaphenalene derivatives of general formula (I) absorb in the ultraviolet radiation range of both type A and type B, said derivatives therefore being useful as UV radiation absorbents. In addition to protect against UV-A radiation and UV-B radiation they can be simultaneously effective in protecting against UV-A and UV-B radiation being still preferably as UV-A radiation protectors and showing a very good UV-A/UV-B ratio (meaning a comparatively high value for UV-A compared to for UV-B).

In addition the heptaazaphenalene derivatives of general formula (I) seem to show very good toxicity profile, good solubility and water improved resistance among other properties that made this compounds became very useful from a formulation point of view.

Another aspect of the present invention are the methods for preparing a heptaazaphenalene derivative in accordance with the first aspect of the invention.

The heptaazaphenalene derivatives of general formula (I) in accordance with the first aspect of the invention can be obtained according to the known procedures (e.g. Shroeder, H.; Kober, E. J. Org. Chem. 1962, 27, 4262). Schematically:

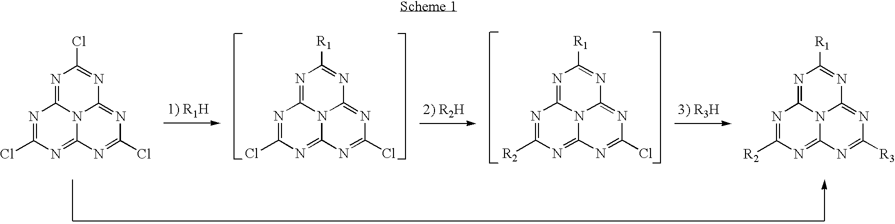

Therefore, in a second aspect the present invention relates to a method for obtaining a heptaazaphenalene derivative of general formula (I) according to the first aspect of the invention,

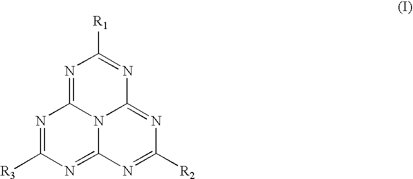

wherein R₁, R₂ and R₃ are the same and represent —NR₅R₆, where R₅ and R₆ are as defined above, which comprises reaction of the 2,5,8-trichloro-1,3,4,6,7,9,9b-heptaazaphenalene derivative of formula (IV) with a derivative of general formula (V)

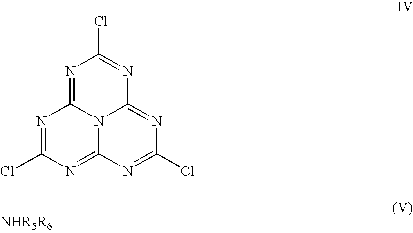

in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent, optionally in the presence of an organic base comprising diisopropylethylamine, triethylamine or pyridine, or an inorganic base comprising potassium carbonate, sodium hydroxide, sodium carbonate, cesium carbonate or sodium bicarbonate.

The process described in the abovementioned second aspect has shown very good possibilities in order to obtain industrial quantities of compounds and leading also to compounds of general formula (I) that show a very good stability and will also give a very good protection against UV-A and UV-B radiation, especially UV-A radiation.

Any of the steps described in the above procedures can also be carried out in a microwave oven, employing a typical procedure of MAOS (microwave assisted organic synthesis). The present invention provides processes of efficiently preparing these compounds in a short time by using microwave irradiation. Microwave assisted chemistry is relatively new compared to some other techniques, however, it has become well established and accepted. Microwave assisted chemical synthesis refers to the use of electromagnetic radiation within the microwave frequencies to provide the energy required to initiate, drive, or accelerate certain chemical reactions. As chemists have long been aware, the application of heat energy is one of the most significant factors in increasing the rate of a wide variety of chemical reactions. Microwave assisted reactions can be completed in a much shorter period of time than conventional thermal-treatment techniques requiring long reaction time. In each of the reactions discussed or illustrated above, pressure is not critical unless otherwise indicated. Pressures from about 0.5 atmospheres to about 5 atmospheres are generally acceptable, and ambient pressure, i.e. about 1 atmosphere, is preferred as a matter of convenience. Under microwave-assisted heating, sealed reactors are indicated, resulting in high-pressure reactions up to as much as 350 psi.

Common microwave equipment may be used in preparation processes according to the present invention. The microwave irradiation may be performed at a power level of 1 to 1600 W, preferably 1 to 300 W, and particularly preferably about 70 W. The duration for the microwave irradiation may vary according to conditions such as the amount or reactant but may be in the range from 20 seconds to 60 minutes, preferably from 1 minute to 20 minutes. The reaction can be carried out at a temperature of 50-280° C., preferably 80-200° C., and more preferably 120-150° C., with of without solvent, under microwave irradiation. A presently preferred microwave furnace is commercially available from CEM, Inc., as model Discover®. The Discover® System incorporates temperature and pressure feedback systems, for example, an infrared temperature sensor positioned below the reaction vessel, for complete control of the reaction. As described above, according to the present invention, heptaazaphenalene derivatives can be prepared within a very short time, i.e. several seconds to several minutes, by microwave irradiation, unlike conventional techniques requiring about 12-50 hours for preparation of compounds for general formula I.

In a third aspect, the present invention relates to a method for obtaining a heptaazaphenalene derivative of general formula (I) according to the first aspect of the invention:

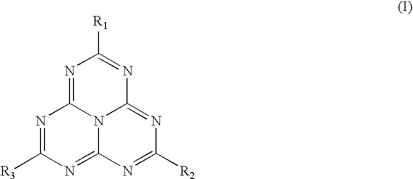

where
R₁, R₂ and R₃ represent —NR₅R₆,
where R₅ and R₆ are as defined above and
wherein one of the radicals R₁, R₂ and R₃ is different from the other two,
characterised in that it includes
a) making the 2,5,8-trichloro-1,3,4,6,7,9,9b-heptaazaphenalene derivative of formula (IV) react with a derivative of general formula (V)

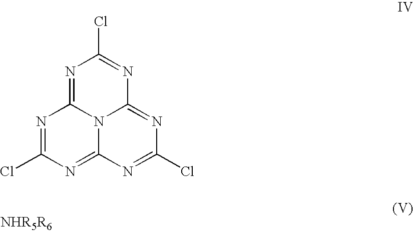

where R₅ and R₆ are as defined above, in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent; optionally in the presence of an organic base comprising diisopropylethylamine, triethylamine or pyridine, or an inorganic base comprising potassium carbonate, sodium hydroxide, sodium carbonate, cesium carbonate or sodium bicarbonate; and
b) adding to the mixture resulting from the preceding stage a second derivative of general formula (V) different from the one used in stage (a) and submitting to reflux.

Any of the steps described above can be conducted trough MAOS (microwave assisted organic synthesis)

In a fourth aspect, the present invention relates to a method for obtaining a heptaazaphenalene derivative of general formula (I) according to the first aspect of the invention:

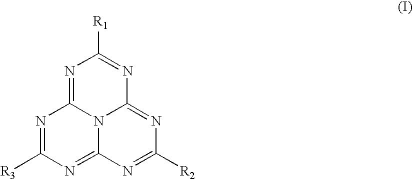

wherein
R₁, R₂ and R₃ are different from each other and represent —NR₅R₆, and
R₅ and R₆ are as defined above,
characterised in that it includes:
a) making the 2,5,8-trichloro-1,3,4,6,7,9,9b-heptaazaphenalene derivative of formula (IV) react with a derivative of general formula (V)

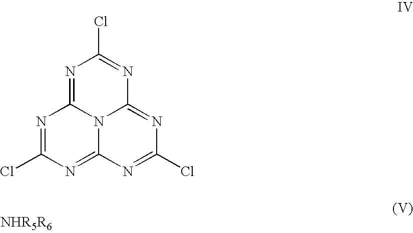

where R₅ and R₆ are as defined above, in a solvent comprising 1,4-dioxane, tetrahydrofuran, toluene, tetrahydrofuran, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent and more preferably between 50° C. and the boiling temperature of the solvent; optionally in the presence of an organic base comprising diisopropylethylamine, triethylamine or pyridine, or an inorganic base comprising potassium carbonate, sodium hydroxide, sodium carbonate, cesium carbonate or sodium bicarbonate;
b) adding to the resulting mixture a derivative of general formula (V) different from the one used in the preceding stage

NHR₅R₆ (V)

where one of R₅ and R₆ are as defined above, and submitting to reflux; and
c) adding to the mixture resulting from stage (b) a derivative of general formula (V) different from the one used in stages (a) and (b)

NHR₅R₆ (V)

where R₅ and R₆ are as defined above.

Any of the steps described above can be conducted trough MAOS (microwave assisted organic synthesis).

Under a fifth aspect, the present invention relates to a method for obtaining a heptaazaphenalene derivative of general formula (I) according to the first aspect of the invention:

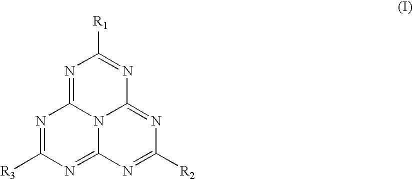

wherein R₁, R₂ and R₃ are the same and represent a derivative of general formula (III)

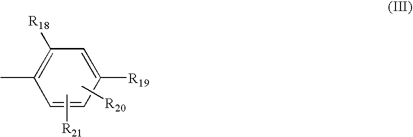

that includes making the 2,5,8-trichloro-1,3,4,6,7,9,9b-heptaazaphenalene derivative of formula (IV) react with a heterocyclic derivative or a compound of general formula (VI)

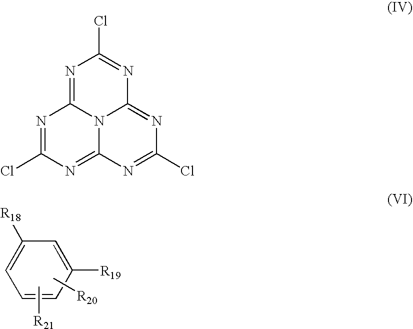

where R₁₈, R₁₉, R₂₀ and R₂₁ are as defined above, in the presence of a Lewis acid comprising, FeCl₃, BF₃, in particular aluminium trichloride, in an inert solvent comprising toluene, 1,1,2,2-tetrachloroethane, tetrahydrofuran, 1,2-dichlorobenzene, nitrobenzene or benzene and at a temperature that ranges between 60° C. and the boiling temperature of the solvent.

Any of the steps described above can be conducted trough MAOS (microwave assisted organic synthesis).

Under a sixth aspect, the present invention relates to a method for obtaining a heptaazaphenalene derivative of general formula (I) according to the first aspect of the invention:

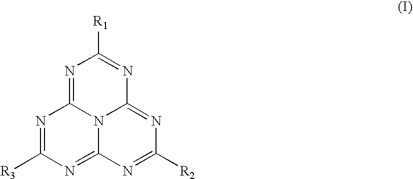

wherein:

one of the radicals R₁, R₂ and R₃ represents an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and

the other two radicals are the same and represent —NR₅R₆, where R₅ or R₆ are as defined in claim 1, which includes the reaction of a derivative of general formula (VII) with a derivative of general formula (V):

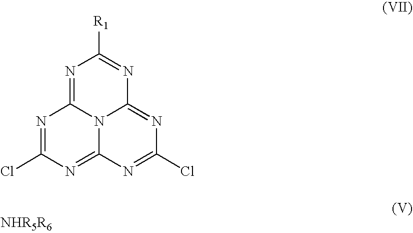

where R₁ is an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and
R₅ and R₆ are as defined above,
in an inert solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent.
Any of the steps described above can be conducted trough MAOS (microwave assisted organic synthesis).

The derivative of general formula (VII) is obtained by reaction of the 2,5,8-trichloro-1,3,4,6,7,9,9b-heptaazaphenalene derivative of general formula (V) with an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S,
in an inert solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent and more preferably between 50° C. and the boiling temperature of the solvent.

Any of the steps described above can be conducted trough MAOS (microwave assisted organic synthesis).

Under a seventh aspect, the present invention relates to a method for obtaining a heptaazaphenalene derivative of general formula (I) according to the first aspect of the invention:

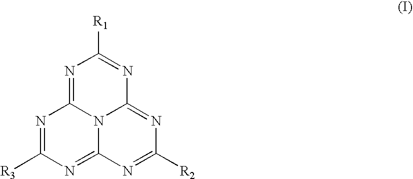

wherein:

two of the radicals R₁, R₂ and R₃ are the same and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and

the third of the radicals R₁, R₂ and R₃ represents —NR₅R₆, where R₅ or R₆ are as defined above,
which includes making the 2,5,8-trichloro-1,3,4,6,7,9,9b-heptaazaphenalene derivative of formula (IV) react with a derivative of general formula (V)

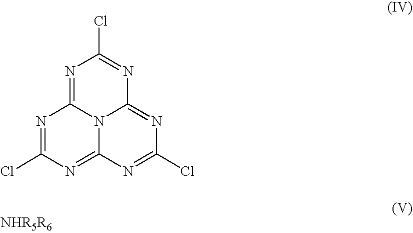

where R₅ and R₆ are as defined above, in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, preferably between room temperature and the boiling temperature of the solvent, and more preferably between 50° C. and the boiling temperature of the solvent, optionally in the presence of an organic base such as diisopropylethylamine, triethylamine or pyridine, or an inorganic base comprising potassium carbonate, sodium hydroxide, sodium carbonate, cesium carbonate or sodium bicarbonate to obtain a compound of general formula (VIII),

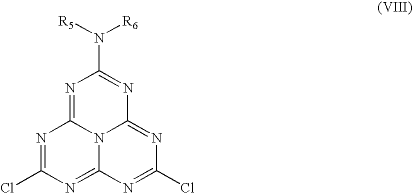

said derivative of general formula (VIII) reacts with a compound of general formula (VI):

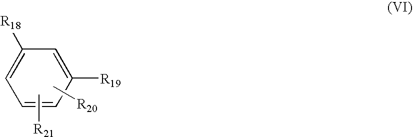

in the presence of a Lewis acid comprising, FeCl₃, BF₃, in particular aluminium chloride, in an inert solvent comprising toluene, xilene, 1,1,2,2-tetrachloroethane, tetrahydrofuran, 1,2-dichlorobenzene, nitrobenzene or benzene and at a temperature between 60° C. and the boiling temperature of the solvent;
thus obtaining the derivative in accordance with the seventh aspect of the invention.

Any of the steps described above can be conducted trough MAOS (microwave assisted organic synthesis).

The compounds of general formula (I), wherein R₂₀ is —SO₃M, where M is as defined above, can be obtained by carrying out, for example, the methods disclosed in U.S. Pat. No. 6,090,370, in particular column 5, line 59-column 6, line 8.

The compounds of general formula (I), wherein an —SO₃M group, where M is as defined above, has been introduced into an alkylic chain, can be obtained according to the methods described in Lewin, G. et al., J. Nat. Prod., 58 (1995) 12, 1840-1847.

The compounds of general formula (I), wherein an —N(R₁₁)₃⁺ group, where R₁₁ is as defined above, has been inserted into an alkylic chain, can be obtained for example by following the methods described in Sharma, M. L. et al., J. Indian Chem. Soc., 74(1997)4, 343-344.

As indicated above, the heptaazaphenalene derivatives of general formula (I) according to the first aspect of the present invention have physicochemical properties such as the absorption of ultraviolet light that allow them to be used as protective agents against UV radiation.

Any of the steps described above can be conducted trough MAOS (microwave assisted organic synthesis).

Also object of the present invention, therefore, are cosmetic, dermatological, veterinary or pharmaceutical formulations or a medicament that include one or more derivatives of general formula (I), according to the first aspect of the invention, and at least one cosmetically, dermatologically or pharmaceutically acceptable carrier or excipient.

A preferred embodiment is a dermatological formulation comprising a compound according to general formula (I)

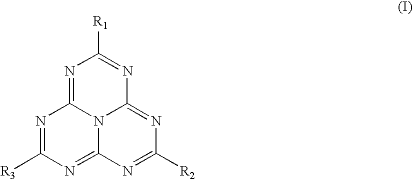

wherein

R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S.

Another preferred embodiment is a cosmetic formulation comprising a compound according to general formula (I):

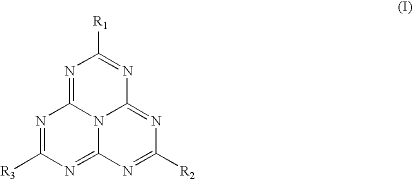

wherein

R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S.

Another preferred embodiment is a pharmaceutical formulation comprising a compound according to general formula (I):

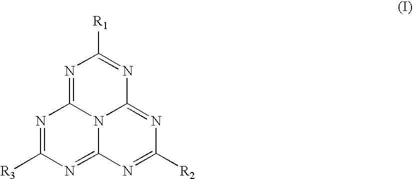

wherein

R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S.
Another preferred embodiment is a veterinary formulation comprising a compound according to general formula (I):

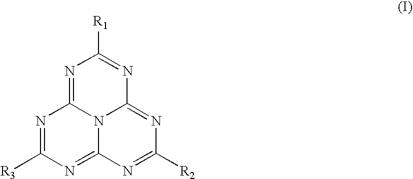

wherein

R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S.
Another preferred embodiment is a medicament comprising a compound according to general formula (I):

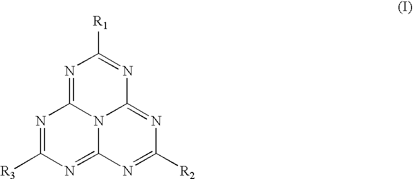

wherein

R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;

R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;

R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S.
In regards to the dermatological, cosmetic, pharmaceutical, veterinary formulation or medicament all heptaazaphenalene compounds described herein and falling under the above definition according to formula I, especially compounds according to the examples and preferred embodiments described above could be comprised in the formulation.
In a preferred embodiment melon and melen

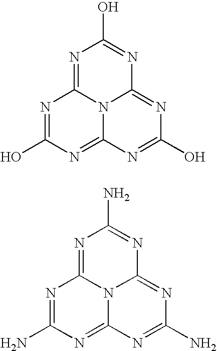

are excluded and thus are not heptaazaphenalene compounds comprised in the formulation.

In a preferred embodiment, said cosmetic, dermatological or pharmaceutical formulation further includes at least one organic, micronized organic or inorganic filter against solar radiation.

In another preferred embodiment said compound according to formula (I) is micronized.

In another preferred embodiment, said formulation further includes at least one active substance.

Said cosmetic, dermatological or pharmaceutical formulation can be adapted for application thereof on the skin and lips in the form of: a non-ionic vesicular dispersion, emulsion, cream, lotion, gel, aerosol, cream-gel, gel-cream, suspension, dispersion, ointment, powder, solid stick, foam, spray, oil, pomade and fluid, among others.

Similarly, said formulation can be adapted for applying it on the hair in the form of a shampoo, lotion, gel, fluid, lacquer, foam, dye, emulsion, cream, spray, among others, and on the nails in the form of a nail varnish, oil and gel, among others.

The organic, micronized organic and inorganic filters are selected from those acceptable under the country's legislation.

The organic filters, for example, can be selected from those approved by the Council of the European Communities (revised text of European Directive 76/768/EEC Annex-7, pages 76-81, published on Oct. 15, 2003) and by the U.S. Food and Drug Administration (see, for example, “Food and Drugs, Sunscreen drug products for over the counter human use”, title 21, volume 5 of Code of Federal Regulations, revised 1 Apr. 2004), such as: anthranilates; camphor derivatives; dibenzoylmethane derivatives; benzotriazole derivatives; diphenylacrylate derivatives; cinnamic derivatives; salycylic derivatives; triazine derivatives such as those disclosed in patents EP-863145, EP-517104, EP-570838, EP-796851, EP-775698 and EP-878469, benzophenone derivatives; benzalmalonate derivatives; benzimidazole derivatives, imidizolines; p-aminobenzoic acid derivatives; polymeric and silicone filters.

The inorganic filters can be selected from a group that includes: metallic oxides as pigments, nanopigments, treated and untreated, such as the dioxide of titanium (amorphous or crystalline), iron, zinc, zirconium or cerium. Moreover, alumina and/or aluminium stearate are conventional coating agents, while examples of untreated metallic oxides as (uncoated) inorganic filters are those described in patents EP518772 and EP518773.

The cosmetic, dermatological and pharmaceutical formulations of the present invention can additionally contain additives and adjuvants that can be selected from fatty acids, organic solvents, thickening agents, softening agents, antioxidants, opacifiers, stabilisers, emollients, hydroxyacids, anti-foaming agents, moisturizing agents, vitamins, fragrances, preservatives, surfactants, sequestering agents, polymers, propellants, acidifying or basifying agents, colorants, dyes, dihydroxyacetone, insect repellent or any other ingredient that is commonly used in cosmetic formulations, and particularly in the production of photoprotective compositions.

Examples of substances/fatty acids include, among others, oils or waxes or mixtures thereof and can include fatty acids, fatty alcohols and fatty acid esters. The oils are advantageously selected from animal and vegetable oils, mineral or synthetic oils, and in particular from liquid petrolatum, liquid paraffin, volatile silicone oils, isoparaffins, polyalphaolefins or fluorated or perfluorated oils. Similarly, the waxes are advantageously selected from animal and vegetable waxes, mineral or synthetic waxes known to skilled in the art.

Examples of organic solvents include short alcohols and polyols.

The thickeners are selected, advantageously, from among acrylic-acid crosslinked polymers, modified and unmodified carob bean rubbers, celluloses and xanthane rubbers, such as hydroxypropylated carob bean rubber, methylhydroxyethylcellulose, hydroxypropylmethylcellulose or hydroxyethylcellulose.

When choosing the excipients, adjuvants, etc., an expert in the subject will ensure that they do not affect the activity of the heptaazaphenalene derivatives of general formula (I) in accordance with the invention.

Under an eighth aspect, the present invention relates to the use of a derivative according to the first aspect of the invention in a cosmetic, dermatological, pharmaceutical or veterinary formulation as a UV radiation filtering agent.

Under a ninth aspect, the present invention relates to the use of a derivative or mixture of derivatives according to the first aspect of the invention for manufacturing a formulation to protect the skin, lips and/or related tissues of a mammal against solar radiation.

Under a tenth aspect, the present invention relates to the use of at least one derivative or mixture of derivatives according to the first aspect of the invention for manufacturing a formulation for preventive use, as a coadjuvant in the treatment of pathologies caused by ultraviolet radiation on the skin, lips and/or related tissues of a mammal, such as polymorphous light eruptions, photoageing, actinic keratasis, vitiligo, urticaria solar, chronic actinic dermatitis and xeroderma pigmentosum. Preferably, said formulation is applied topically.

In a preferred embodiment said mammal is a human.

The properties of the heptaazaphenalene derivatives of general formula (I) mean that said compounds are also useful as photostabilisers of polymers and as solar filters for textile fibres.

In the present invention, “polymers” means chemical compounds of natural or synthetic origin and generally of high molecular weight made up of structural units (monomers) linked to each other by means of covalent bonds. Examples of polymers include but are not limited to proteins, polysaccharides, cellulose, natural rubber, nucleic acids, polyethylene, polycarbonates, silicone polymers, polyurethanes, polyesters, polyamides and acrylic polymers, among others.

There follow some examples where the UV λₘₐₓ and εₘₐₓ have been measured according to general methods known for the person skilled in the art by way of non-restrictive illustration of the present invention.

### EXAMPLES

### Example 1

### Synthesis of 2,5,8-tris-(4-(butoxycarbonyl)phenylamino)-1,3,4,6,7,9,9b-heptaazaphenalene

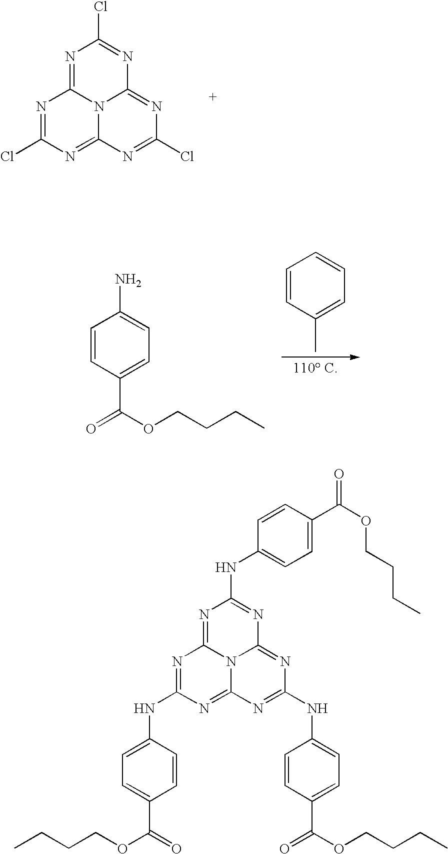

A mixture of 2,5,8-trichloro-1,3,4,6,7,9,9b-heptaazaphenalene (50 mg, 0.18 mmol) and butyl 4-aminobenzoate (210 mg, 1.08 mmol) in toluene (2 mL) is refluxed for 30 minutes. The resulting solid is filtered by porous plate and washed with toluene (10 mL), to yield 2,5,8-tris-(4-(butoxycarbonyl)phenylamino)-1,3,4,6,7,9,9b-heptaazaphenalene (130 mg, 0.17 mmol, 96%).

M.P. 289-290° C.

¹H NMR (300 MHz, DMSO-d₆): δ 0.95 (t, J=7.2 Hz, 9H), 1.41 (m, 6H), 1.65 (m, 6H), 4.20 (m, 6H), 7.92 (m, 12H), 10.80 (m, 3H).

MS-EI (m/z): 747 (M+1).

UV λₘₐₓ=325 nm; εₘₐₓ=100000 M⁻¹ cm⁻¹ (CHCl₃—EtOH).

### Example 2

### Synthesis of 2,5,8-tris-(4-(2-ethylhexyloxycarbonyl)phenylamino)-1,3,4,6,7,9,9b-heptaazaphenalene

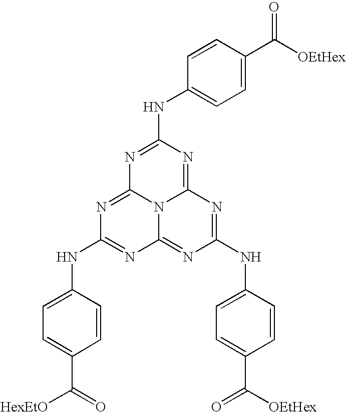

A mixture of 2,5,8-trichloro-1,3,4,6,7,9,9b-heptaazaphenalene (50 mg, 0.18 mmol) and 2-ethylhexyl 4-aminobenzoate (270 mg, 1.08 mmol) in toluene (2 mL) is refluxed for 1 hour. The solvent is evaporated in vacuo and the crude product is purified by silica gel column chromatography, eluting with hexane/ethyl acetate 2/1. 2,5,8-tris-(4-(2-ethylhexyloxycarbonyl)phenylamino)-1,3,4,6,7,9,9b-heptaazaphenalene is obtained (160 mg, 0.17 mmol, 97%).

M.P. 183-186° C.

¹H NMR (300 MHz, DMSO-d₆): δ 0.93 (m, 18H), 1.30 (m, 24H), 1.62 (m, 3H), 4.18 (d, J=5.4 Hz, 6H), 7.95 (m, 12H), 10.85 (m, 3H).

MS-EI (m/z): 916 (M+1).

UV: λₘₐₓ=326 nm; εₘₐₓ=95000 M⁻¹ cm⁻¹ (CHCl₃).

### Example 3

### Synthesis of 2,5,8-tris-(4-(imidazo[1,2-a]pyridin-2-yl)-phenylamino)-1,3,4,6,7,9,9b-heptaazaphenalene

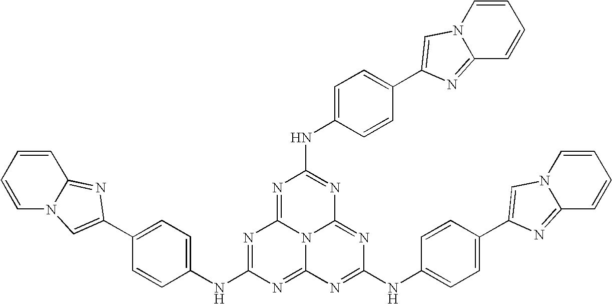

A mixture of 2,5,8-trichloro-1,3,4,6,7,9,9b-heptaazaphenalene (25 mg, 0,09 mmol), 4-(H-imidazo[1,2-a]pyrridin-2-yl)benzenamine (114 mg, 0.54 mmol) and diisopropylamine (155 μL, 0.9 mmol) in toluene (2 mL) is refluxed for 1 hour. N-methylpyrrolidone (0.2 mL) is added and heated to 120° C. for 2 hours. The system is then allowed to cool. The solid is filtered by a porous plate and purified by silica gel column chromatography, eluting with mixtures of ethyl acetate/methanol. This yields 2,5,8-tris-(4-(imidazo[1,2-a]pyrridin-2-yl)-phenylamino)-1,3,4,6,7,9,9b-heptaazaphenalene (31 mg, 0.03 mmol, 22%).

¹H NMR (300 MHz, DMSO-d₆): δ 6.90 (m, 3H), 7.30 (m, 3H), 7.60 (m, 3H), 7.90 (m, 12H), 8.38 (s, 3H), 8.55 (m, 3H), 10.65 (m, 3H).

MS-EI (m/z): 795 (M+1).

UV: λₘₐₓ=353 nm; εₘₐₓ=75000 M⁻¹ cm⁻¹ (DMSO).

### Example 4

### Synthesis of 2,5,8-tris-(4-(4,5,6,7-tetrahydro-2,6,6-trimethyl-4-oxoindol-1-yl)phenylamine)-1,3,4,6,7,9,9b-heptaazaphenalene

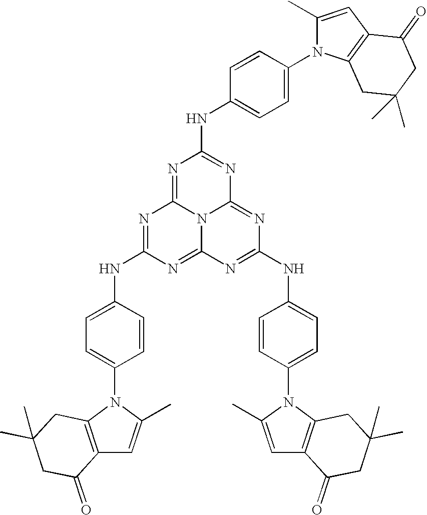

A mixture of 2,5,8-trichloro-1,3,4,6,7,9,9b-heptaazaphenalene (50 mg, 0.18 mmol) and 1-(4-aminophenyl)-6,7-dihydro-2,6,6-trimethyl-1H-indol-4(5H)-one (291 mg, 1.08 mmol) in toluene (2 mL) is refluxed for 4 hours. The system is then allowed to cool. The solid is filtered by a porous plate and washed with HCl 1N (10 mL), H₂O (10 mL) and Et₂O (10 mL). The crude product obtained is purified by silica gel column chromatography, eluting with mixtures of hexane/ethyl acetate. This yields 2,5,8-tris-(4-(4,5,6,7-tetrahydro-2,6,6-trimethyl-4-oxoindol-1-yl)phenylamine)-1,3,4,6,7,9,9b-heptaazaphenalene (98 mg, 0.1 mmol, 56%).

¹H NMR (300 MHz, CDCl₃): δ 1.05 (s, 18H), 2.10 (s, 9H), 2.38 (m, 12H), 6.40 (s, 3H), 7.20 (m, 6H), 7.80 (m, 9H).

MS-EI (m/z): 973 (M+1).

UV: λₘₐₓ=314 nm; εₘₐₓ=103000 M⁻¹ cm⁻¹ (EtOH).

### Example 5

### Synthesis of 2,5,8-tris-(biphenyl-4-ylamino)-1,3,4,6,7,9,9b-heptaazaphenalene

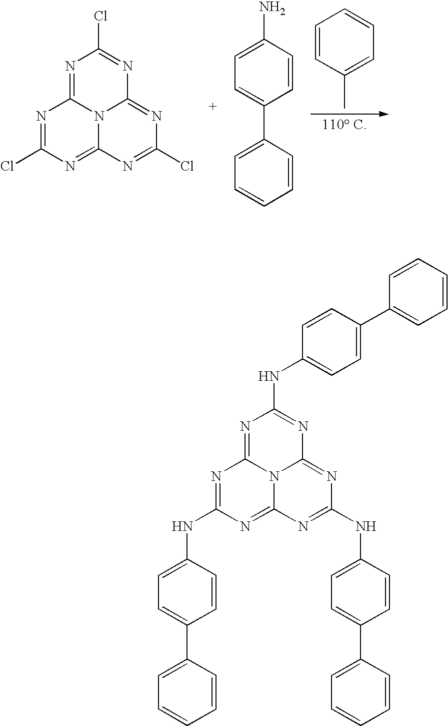

A mixture of 2,5,8-trichloro-1,3,4,6,7,9,9b-heptaazaphenalene (50 mg, 0.18 mmol) and 4-aminobiphenyl (183 mg, 1.08 mmol) in toluene (2 mL) is refluxed for 2 hours. The system is then allowed to cool. The solid is filtered by a porous plate and washed with HCl 1N (10 mL), H₂O (10 mL) and Et₂O (10 mL). This yields 2,5,8-tris-(biphenyl-4-ylamino)-1,3,4,6,7,9,9b-heptaazaphenalene.

M.P.>310° C.

¹H NMR (300 MHz, DMSO-d₆): δ 7.25 (m, 3H), 7.40 (m, 6H), 7.65 (m, 12H), 7.80 (m, 6H), 10.60 (m, 3H). MS-EI (m/z): 675 (M+1).

### Example 6

### Synthesis of 2,5,8-tris-(biphenyl-4-ylamino)-1,3,4,6,7,9,9b-heptaazaphenalene

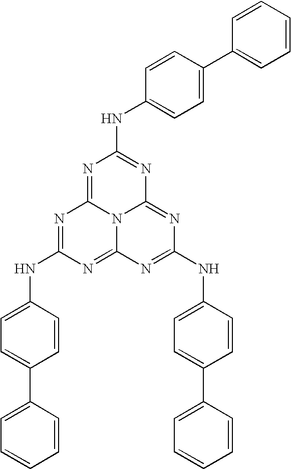

Following the method described on example 1. Yield: 90%. M.P.>310° C.

¹H NMR (300 MHz, DMSO-d₆): δ 7.25 (m, 3H), 7.40 (m, 6H) 7.65 (m, 12H), 7.80 (m, 6H), 10.60 (s, 3H)

MS-EI (m/z): 675 (M+1).

UV λₘₐₓ=333 nm; εₘₐₓ=103000 M⁻¹ cm⁻¹ (DMSO).

### Example 7

### Synthesis of 2,5,8-tris-(4-(1H-benzo[d]imidazol-2-yl)phenylamino)-1,3,4,6,7,9,9b-heptaazaphenalene

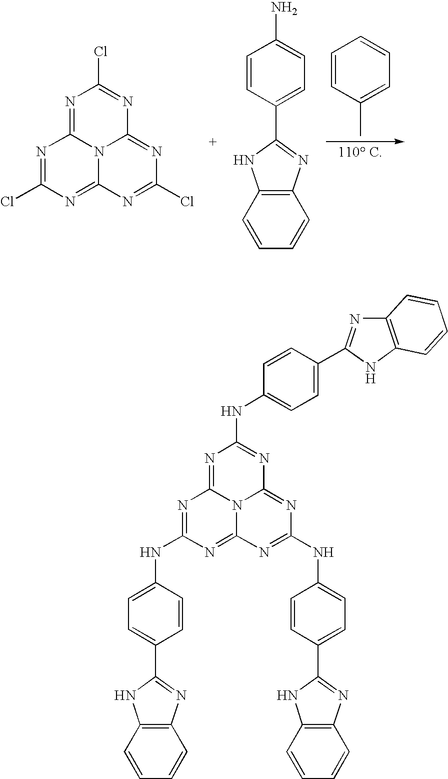

A mixture of 2,5,8-trichloro-1,3,4,6,7,9,9b-heptaazaphenalene (50 mg, 0.18 mmol) and 4-(1H-benzo[d]imidazol-2-yl)phenylamine (227 mg, 1.08 mmol) in toluene (2 mL) is refluxed for 5 hours. The system is then allowed to cool. The solid is filtered by a porous plate and washed with HCl 1N (10 mL), H₂O (10 mL), MeOH (10 mL) and Et₂O (10 mL). This yields 2,5,8-tris-(4-(1H-benzo[d]imidazol-2-yl)phenylamino)-1,3,4,6,7,9,9b-heptaazaphenalene.

NMR¹H (300 MHz, DMSO-d₆): δ 7.40 (m, 6H), 7.65 (m, 6H), 8.05 (m, 6H), 8.30 (m, 6H). EM-IE (m/z): 795 (M+1).

UV λₘₐₓ=358 nm); εₘₐₓ=107000 M⁻¹ cm⁻¹ (CHCl₃-MeOH).

### Example 8

### Synthesis of 2,5,8-tris-(4-(1H-benzo[d]imidazol-2-yl)phenylamino)-1,3,4,6,7,9,9b-heptaazaphenalene

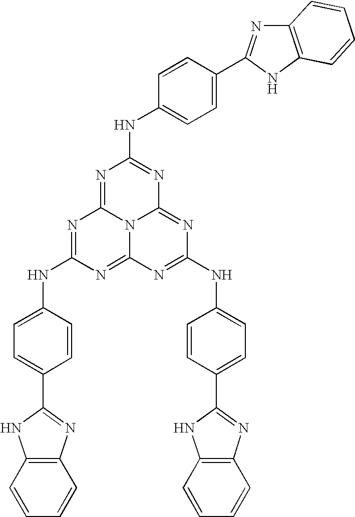

Following the method described on example 1. Yield: 82%.

M.P.>275° C.

¹H NMR (300 MHz, DMSO-d₆): δ 7.40 (m, 6H), 7.65 (m, 6H), 8.05 (m, 6H), 8.30 (m, 6H), 10.90 (m, 3H).

MS-EI (m/z): 795 (M+1).

UV λₘₐₓ=358 nm; εₘₐₓ=107000 M⁻¹ cm⁻¹ (CHCl₃-MeOH)

### Example 9

### Synthesis of 2,5-bis-(biphenyl-4-ylamino)-8-(4-(butoxycarbonyl)phenylamino)-1,3,4,6,7,9,9b-heptaazaphenalene

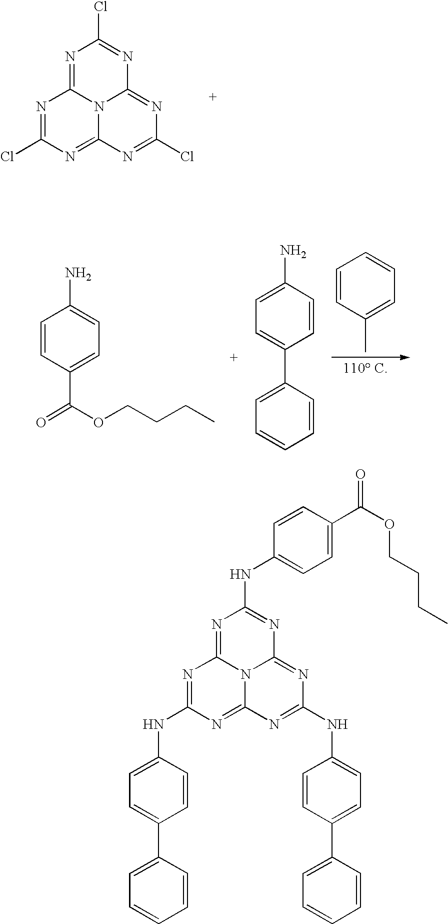

A mixture of 2,5,8-trichloro-1,3,4,6,7,9,9b-heptaazaphenalene (50 mg, 0.18 mmol) and butyl 4-aminobenzoate (38 mg, 0.19 mmol) in toluene (2 mL) is refluxed for 1 hour. The mixture is then cooled, 4-aminobiphenyl (122 mg, 0.72 mmol) is added and heated again at reflux for a further 1 hour. The solid is filtered by a porous plate and washed with toluene (10 mL), to yield 2,5-bis-(biphenyl-4-ylamino)-8-(4-(butoxycarbonyl)phenylamino)-1,3,4,6,7,9, 9b-heptaazaphenalene.

MS-EI (m/z): 699 (M+1).

### Example 10

### Synthesis of 2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene

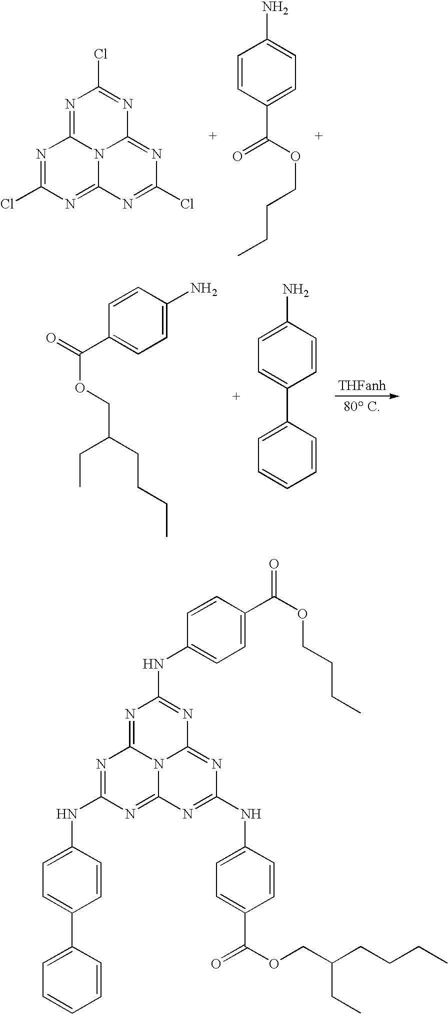

A mixture of 2,5,8-trichloro-1,3,4,6,7,9,9b-heptaazaphenalene (50 mg, 0.18 mmol) and butyl 4-aminobenzoate (38 mg, 0.19 mmol) in THF (2 mL) is refluxed for 1 hour. The mixture is then cooled, 2-ethylhexyl aminobenzoate (50 mg, 0.19 mmol) is added and heated at reflux for a further 3 hours. The mixture is then cooled, 4-aminobiphenyl (122 mg, 0.72 mmol) is added and it is again heated at reflux for a further 3 hours. The resulting solid is filtered by porous plate and washed with MeOH (10 mL), to yield 2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene.

MS-EI (m/z): 779 (M+1).

### Example 11

### Synthesis of 2,5,8-tris-(2,4-dihydroxyphenyl)-1,3,4,6,7,9,9b-heptaazaphenalene

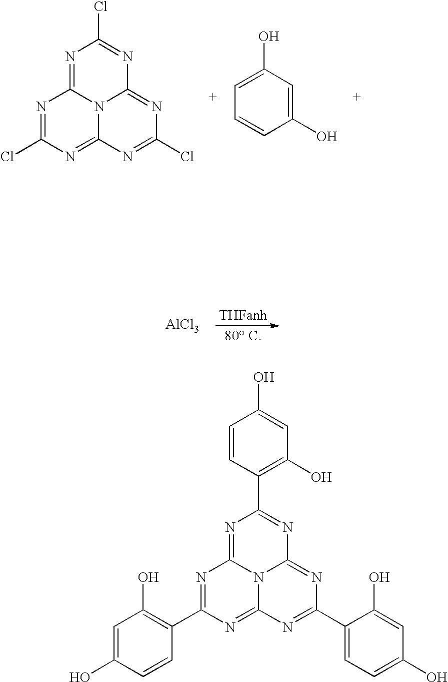

A mixture of 2,5,8-trichloro-1,3,4,6,7,9,9b-heptaazaphenalene (50 mg, 0.18 mmol), resorcinol (66 mg, 0.59 mmol) and aluminium trichloride (79 mg, 0.59 mmol) in THF (2 mL) is heated at reflux. After 5 hours, the system is cooled and HCl 1N (2 mL) is added and left under stirring for 10 minutes. The solvent is evaporated in vacuo and extracted with AcOEt (3×10 mL). The combined organic phases are washed with saturated solution of NaCl (1×10 mL), dried over Na₂SO₄ and the solvent eliminated in vacuo. This yields 2,5,8-tris-(2,4-dihydroxyphenyl)-1,3,4,6,7,9,9b-heptaazaphenalene.

Yield: 67%.

¹H NMR (300 MHz, DMSO-d₆): δ 6.31 (d, J=2 Hz, 3H), 6.49 (dd, J=9, 2 Hz, 3H), 8.13 (d, J=9 Hz, 3H), 10.90 (s, 3H), 12.97 (m, 3H).

HPLC-MS (m/z): 497 (M+).

UV: λₘₐₓ=394 nm; εₘₐₓ=56000 M⁻¹ cm⁻¹ (DMSO).

### Example 12

### a) Synthesis of 2,5-dichloro-8-(1-methyl-1H-pyrrol-2-yl)-1,3,4,6,7,9,9b-heptaazaphenalene

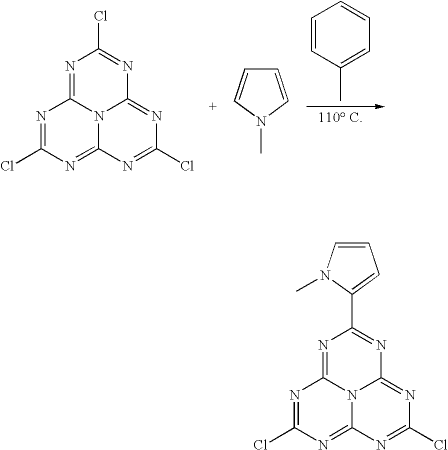

A mixture of 2,5,8-trichloro-1,3,4,6,7,9,9b-heptaazaphenalene (50 mg, 0.18 mmol) and 1-methylpyrrol (16 μL, 0.18 mmol) in toluene (2 mL) is refluxed for 4 hours. The system is then left to cool. The solid is filtered by porous plate and washed with dry toluene (10 mL). This yields 2,5-dichloro-8-(1-methyl-1H-pyrrol-2-yl)-1,3,4,6,7,9,9b-heptaazaphenalene (41 mg, 0.12 mmol, 71%).

¹H NMR (300 MHz, CDCl₃): δ 4.10 (s, 3H), 6.38 (m, 1H), 7.15 (m, 1H), 7.65 (m, 1H).

MS-EI (m/z): 319 (M−2).

### b) Synthesis of 2,5-bis-(4-(butoxycarbonyl)phenylamino)-8-(2-(1-methyl-1H-pyrrol-2-yl)-1,3,4,6,7,9,9b-heptaazaphenalene

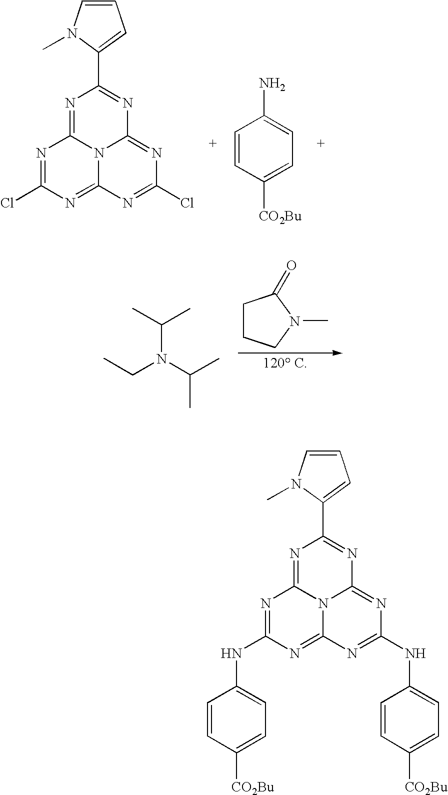

A mixture of 2,5-dichloro-8-(1-methyl-1H-pyrrol-2-yl)-1,3,4,6,7,9,9b-heptaazaphenalene (41 mg, 0.12 mmol), butyl 4-aminobenzoate (98 mg, 0.51 mmol) and diisopropylethylamine (110 μL, 0.63 mmol) in N-methylpyrrolidone (0.5 mL) is heated for 7 hours at 120° C. The system is then left to cool and H₂O (5 mL) is added. The solid is filtered by porous plate and washed with Et₂O (10 mL). This yields 2,5-bis-(4-(butoxycarbonyl)phenylamino)-8-(2-(1-methyl-1H-pyrrol-2-yl)-1,3,4,6,7,9,9b-heptaazaphenalene (62 mg, 0.09 mmol, 76%).

M.P. 195-196° C.

¹H NMR (300 MHz, CDCl₃): δ 0.90 (m, 6H), 1.85 (m, 8H), 3.80 (s, 3H), 4.25 (m, 4H), 6.10 (m, 1H), 6.85 (m, 1H), 7.65 (m, 4H), 7.90 (m, 1H), 7.95 (m, 4H), 8.30 (m, 2H).

MS-EI (m/z): 635 (M+1).

### Example 13

### Synthesis of 2-(4-(carboxy)phenylamino)-5,8-bis-(4-methylphenyl)-1,3,4,6,7,9,9b-heptaazaphenalene

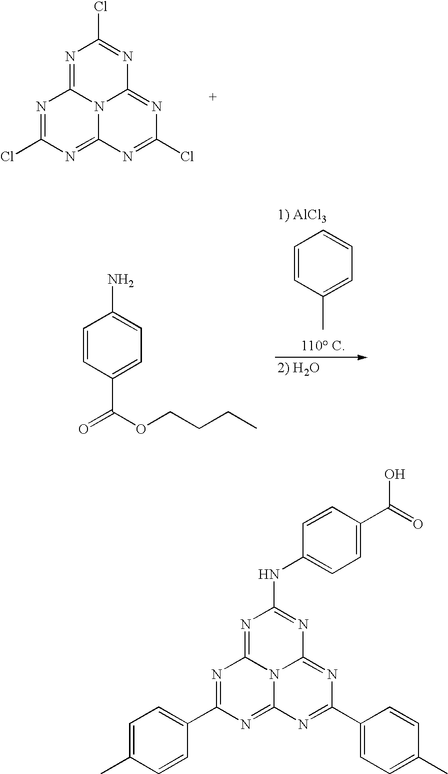

A mixture of 2,5,8-trichloro-1,3,4,6,7,9,9b-heptaazaphenalene (50 mg, 0.18 mmol) and butyl 4-aminobenzoate (38 mg, 0.19 mmol) in toluene (2 mL) is refluxed for 1 hour. The mixture is then cooled, aluminium trichloride (48 mg, 0.36 mmol) is added and heated again at reflux. After 3 hours, H₂O (5 mL) is added and left at reflux for 30 minutes. The system is cooled and the resulting solid is filtered by porous plate. The part insoluble in CH₂Cl₂ corresponds to 2-(4-(carboxy)phenylamino)-5,8-bis-(4-methylphenyl)-1,3,4,6,7,9,9b-heptaazaphenalene.

MS-EI (m/z): 489 (M+1).

### Example 14

### Synthesis of 2,5,8-tris-(4-benzoylphenylamino)-1,3,4,6,7,9,9b-heptaazaphenalene

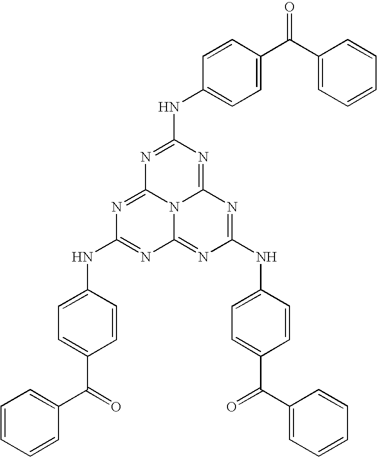

Following the method described in example 1. Yield: 93%.

M.P. >270° C.

¹H NMR (300 MHz, DMSO-d₆): δ 7.45 (m, 6H), 7.65 (m, 15H), 7.90 (m, 6H), 10.90 (m, 3H).

MS-EI (m/z): 759 (M+1).

UV: λₘₐₓ=339 nm; εₘₐₓ=112000 M⁻¹ cm⁻¹ (DMSO).

### Example 15

### Synthesis of 2,5,8-tris-(4-(1H-pyrazol-1-yl)phenylamino)-1,3,4,6,7,9,9b-heptaazaphenalene

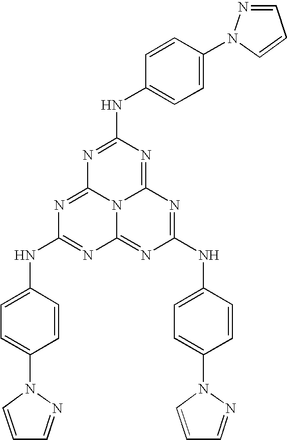

Following the method described in example 1. Yield: 81%.

M.P. 224-227° C.

¹H NMR (300 MHz, DMSO-d₆): δ 6.50 (s, 3H), 7.80 (m, 15H), 8.40 (s, 3H), 10.60 (s, 3H).

MS-EI (m/z): 645 (M+1).

UV λₘₐₓ=330 nm; εₘₐₓ=99000 M⁻¹ cm⁻¹ (DMSO).

### Example 16

### Synthesis of 2,5,8-tris-(4-butoxy-2-hydroxyphenyl)-1,3,4,6,7,9,9b-heptaazaphenalene

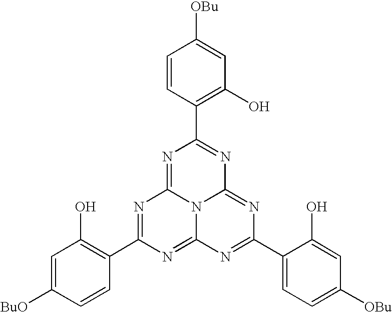

Following the method described in example 9.

MS-EI (m/z): 666 (M+1).

### Example 17

### Synthesis of 2,5,8-tris-(naphthalen-2-ylamino)-1,3,4,6,7,9,9b-heptaazaphenalene

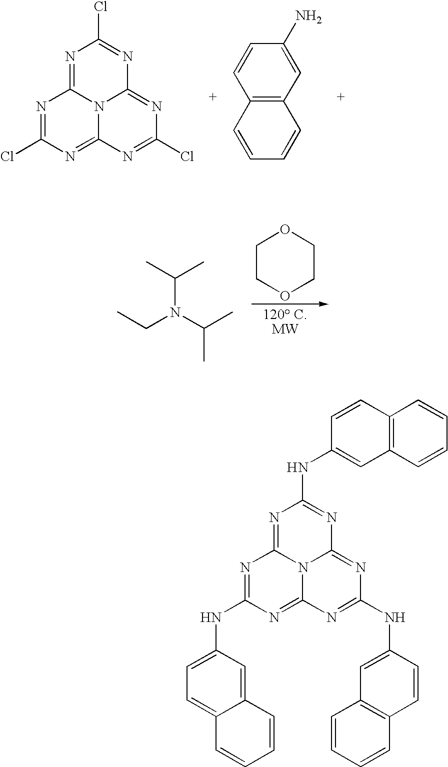

A mixture of 2,5,8-trichloro-1,3,4,6,7,9,9b-heptaazaphenalene (50 mg, 0.18 mmol), naphthalene-2-amine (155 mg, 1.08 mmol) and diisopropylamine (247 μL, 1.44 mmol) in 1,4-dioxane (2 mL) is heated at 120° C. for 10 minutes in a MW oven. The system is cooled. The solid is filtered by a porous plate and washed with 1,4-dioxane and methanol. This yields 2,5,8-tris-(naphthalen-2-ylamino)-1,3,4,6,7,9,9b-heptaazaphenalene (85 mg, 79%).

M.P.>275° C.

¹H NMR (300 MHz, DMSO-d₆): δ 7.40 (m, 6H), 7.50 (m, 6H), 7.90 (m, 12H), 8.35 (m, 6H), 10.70 (m, 3H).

MS-EI (m/z): 597 (M+1).

UV: λₘₐₓ=279 nm; εₘₐₓ=89000 M⁻¹ cm⁻¹; λₘₐₓ=331 nm; εₘₐₓ=75000 M⁻¹ cm⁻¹ (DMSO).

### Example 18

### Synthesis of 2,5-bis-(4-(butoxycarbonyl)phenylamino)-8-(2-(1-methyl-1H-indol-3-yl)-1,3,4,6,7,9,9b-heptaazaphenalene

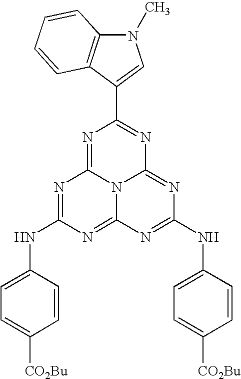

Following the method described in example 11. Yield: 84%.

MS-EI (m/z): 685 (M+1).

UV: λₘₐₓ=337 nm; εₘₐₓ=65000 M⁻¹ cm⁻¹ (DMSO).

### Example 19

### Synthesis of 2,5,8-tris-(biphenyl-4-yloxy)-1,3,4,6,7,9,9b-heptaazaphenalene

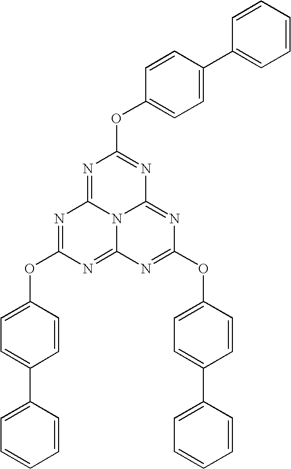

Following the method described in example 16. Yield: 79%.

¹H NMR (300 MHz, DMSO-d₆): δ 7.25 (m, 6H), 7.35 (m, 3H), 7.45 (m, 6H), 7.65 (m, 12H).

MS-EI (m/z): 678 (M+1).

UV: λₘₐₓ=262 nm; εₘₐₓ=81000 M⁻¹ cm⁻¹ (DMSO).

### Example 20

### Synthesis of 2,5,8-tris-(3-methoxyphenylamino)-1,3,4,6,7,9,9b-heptaazaphenalene

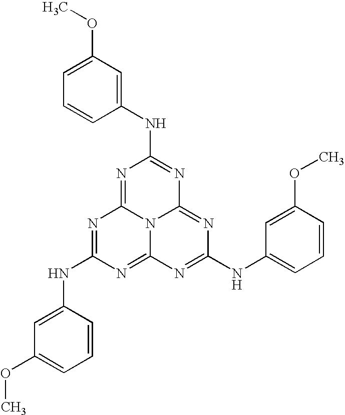

Following the method described in example 16. Yield: 82%.

M.P.>275° C.

¹H NMR (300 MHz, DMSO-d₆): δ 3.75 (s, 9H), 6.64 (d, J=7 Hz, 3H), 7.45 (m, 6H), 10.35 (m, 3H).

MS-EI (m/z): 537 (M+1).

UV: λₘₐₓ=312 nm; εₘₐₓ=62000 M⁻¹ cm⁻¹ (MeOH).

### Example 21

### Synthesis of 2,5,8-tris-(4-methoxyphenylamino)-1,3,4,6,7,9,9b-heptaazaphenalene

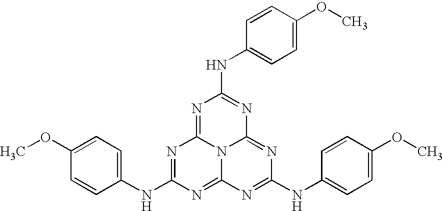

Following the method described in example 16. Yield: 75%.

¹H NMR (300 MHz, DMSO-d₆): δ 3.72 (s, 9H), 6.89 (d, J=8.4 Hz, 6H), 7.64 (d, J=8.4 Hz, 6H), 10.20 (m, 3H).

MS-EI (m/z): 537 (M+1).

UV: λₘₐₓ=324 nm; εₘₐₓ=59000 M⁻¹ cm⁻¹ (DMSO).

### Example 22

### Synthesis of 2,5,8-tris-(4-((E)-3-ethoxy-3-oxoprop-1-enyl)phenylamino)-1,3,4,6,7,9,9b-heptaazaphenalene

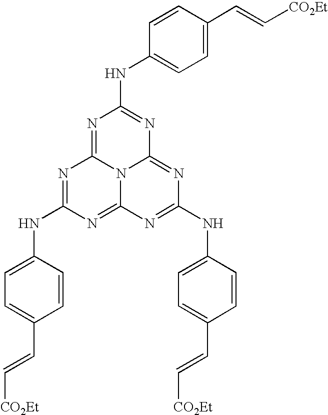

Following the method described in example 16. Yield: 76%.

M.P.>275° C.

¹H NMR (300 MHz, DMSO-d₆): δ 1.25 (m, 9H), 4.20 (m, 8H), 6.55 (m, 3H), 7.60 (m, 3H), 7.65 (m, 6H), 7.80 (m, 6H), 10.70 (m, 3H).

MS-EI (m/z): 741 (M+1).

UV: λₘₐₓ=359 nm; εₘₐₓ=135900 M⁻¹ cm⁻¹ (DMSO).

### Example 23

### Synthesis of 2,5,8-tris-(methoxycarbonyl-4′-biphenyl-4-ylamino)-1,3,4,6,7,9,9b-heptaazaphenalene

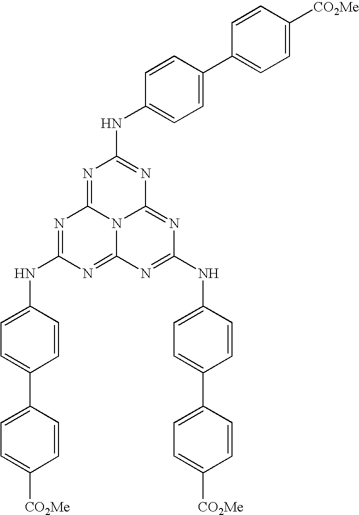

Following the method described in example 16. Yield: 66%.

¹H NMR (300 MHz, DMSO-d₆): δ 3.80 (s, 9H), 7.90 (m, 24H), 10.80 (m, 3H).

MS-EI (m/z): 849 (M+1).

UV: λₘₐₓ=346 nm; εₘₐₓ=99000 M⁻¹ cm⁻¹ (DMSO).

### Example 24

### Synthesis of 2,5,8-tris-(4-(methoxycarbonyl)phenylamino)-1,3,4,6,7,9,9b-heptaazaphenalene

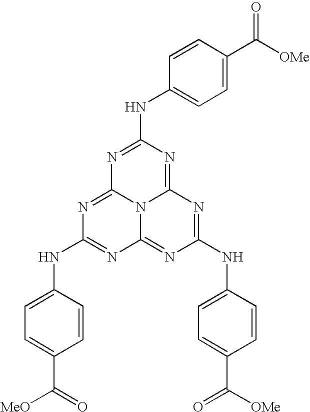

Following the method described in example 1. Yield: 59%.

M.P. 288-291° C.

¹H NMR (300 MHz, DMSO-d₆): δ 3.70 (s, 9H), 7.92 (m, 12H)

UV: λₘₐₓ=329 nm; εₘₐₓ=116000 M⁻¹ cm⁻¹ (DMSO).

### Example 25

### Synthesis of 2,5,8-tris-(4-methylphenyl)-1,3,4,6,7,9,9b-heptaazaphenalene

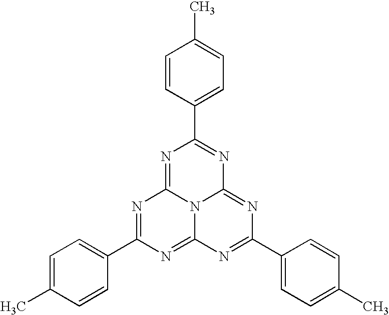

Following the method described in example 9.

MS-EI (m/z): 444 (M+1).

UV: λₘₐₓ=327 nm; εₘₐₓ=86000 M⁻¹ cm⁻¹ (DMSO).

### Example 26

### Synthesis of 2,5,8-tris-(4-(1H-benzo[d]imidazol-2-yl)-3-hydroxyphenylamino)-1,3,4,6,7,9,9b-heptaazaphenalene

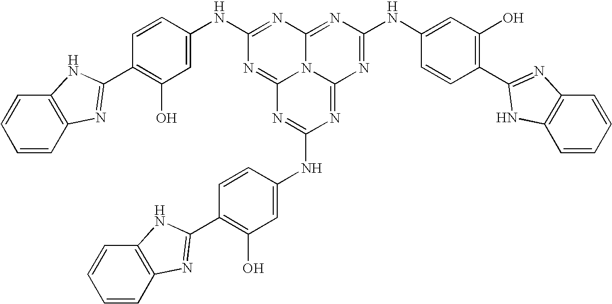

Following the method described in example 1. Yield: 44%.

¹H NMR (300 MHz, DMSO-d₆): δ 7.20 (s, 9H), 7.60 (m, 9H), 7.90 (m, 3H), 13.20 (m, 3H).

MS-EI (m/z): 842 (M).

UV: λₘₐₓ=354 nm, εₘₐₓ=122000 M⁻¹ cm⁻¹; εₘₐₓ=368 nm; εₘₐₓ=123000 M⁻¹ cm⁻¹ (DMSO).

### Example 27

### Synthesis of 2,5,8-tris-(4-(phenylamino)phenylamino)-1,3,4,6,7,9,9b-heptaazaphenalene

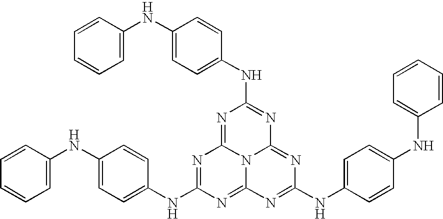

Following the method described in example 1. Yield: 94%.

¹H NMR (300 MHz, DMSO-d₆): ε 6.77 (m, 6H), 7.00 (m, 9H), 7.15 (m, 6H), 7.55 (m, 6H), 8.01 (s, 3H), 10.20 (m, 3H).

MS-EI (m/z): 720 (M+1).

UV: λₘₐₓ=360 nm, εₘₐₓ=60000 M⁻¹ cm⁻¹ (DMSO).

### Example 28

### Synthesis of 2,5,8-tris-((4-(E)-styrylphenyl)amino)-1,3,4,6,7,9,9b-heptaazaphenalene

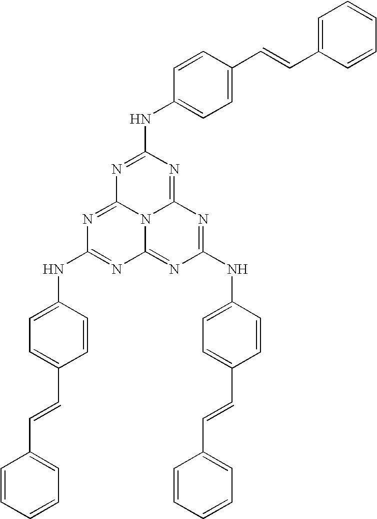

Following the method described in example 1. Yield: 76%. M.P.>270° C.

¹H NMR (300 MHz, DMSO-d₆): δ 7.20 (m, 9H), 7.35 (m, 6H), 7.60 (m, 12H), 7.80 (m, 6H), 10.60 (m, 3H).

UV: λₘₐₓ=367 nm; εₘₐₓ=163000 M⁻¹ cm⁻¹ (DMSO).

### Example 29

### Synthesis of 2,5,8-tris-(2-ethylhexylamino)-1,3,4,6,7,9,9b-heptaazaphenalene

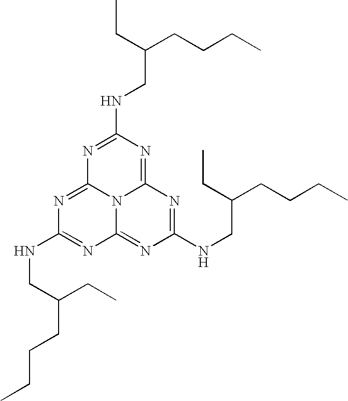

Following the method described in example 1. Yield: 57%.

¹H NMR (300 MHz, DMSO-d₆): δ 0.90 (m, 18H), 1.35 (m, 24H), 1.60 (m, 3H), 3.30 (m, 6H).

UV: λₘₐₓ=265 nm; εₘₐₓ=92000 M⁻¹ cm⁻¹ (DMSO).

### Example 30

### Synthesis of 2,5,8-tris-(4-(L-menthylcarbonyl)phenylamino)-1,3,4,6,7,9,9b-heptaazaphenalene

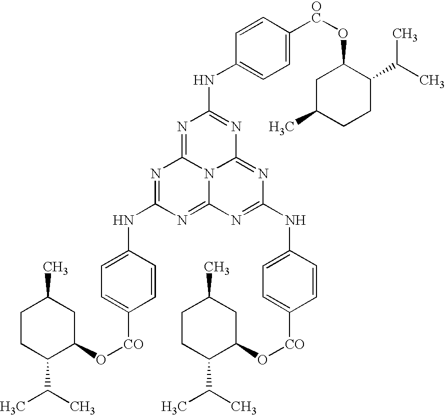

Following the method described in example 1. Yield: 49%.

M.P. 202-206° C.

¹H NMR (300 MHz, DMSO-d₆): δ 0.72 (d, J=7 Hz, 9H), 0.85 (m, 21H), 1.10 (m, 6H), 1.50 (m, 6H), 1.70 (m, 6H), 1.82 (m, 3H), 2.00 (m, 3H), 4.88 (dt, J=6.5, 4.2 Hz, 3H), 7.90 (m, 12H), 10.90 (m, 3H).

UV: λₘₐₓ=326 nm; εₘₐₓ=118000 M⁻¹ cm⁻¹ (DMSO).

### Example 31

### Synthesis of 2,5,8-tris-(4-((3,3,5-trimethylcyclohexyloxy)carbonyl)phenylamino)-1,3,4,6,7,9,9b-heptaazaphenalene

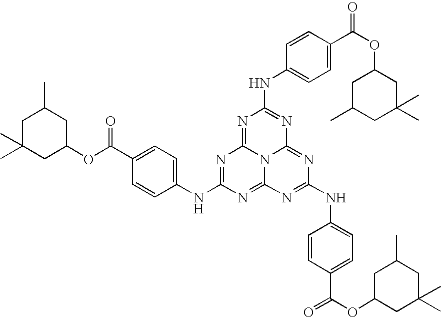

Following the method described in example 1. Yield: 92%.

M.P.>275° C.

¹H NMR (300 MHz, DMSO-d₆): δ 0.90 (d, J=7.2 Hz, 18H), 1.01 (m, 15H), 1.20 (m, 3H), 1.30 (m, 3H), 1.70 (m, 6H), 2.00 (m, 3H), 4.88 (m, 3H), 7.88 (m, 12H), 10.85 (m, 3H).

UV: λₘₐₓ=329 nm; εₘₐₓ=143000 M⁻¹ cm⁻¹ (DMSO).

### Example 32

### Synthesis of 2,5,8-tris-(4-((E)-3-(2-ethylhexyloxy)-3-oxoprop-1-enyl)phenylamino)-1,3,4,6,7,9,9b-heptaazaphenalene

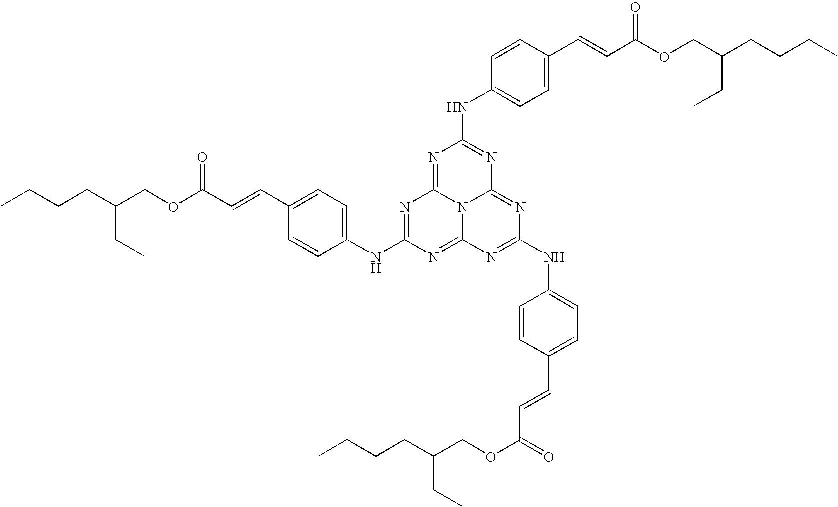

Following the method described in example 1. Yield: 90%.

M.P. 213-216° C.

¹H NMR (300 MHz, DMSO-d₆): δ 0.86 (t, J=7.2 Hz, 18H), 1.27 (m, 32H), 1.57 (m, 3H), 4.04 (d, J=4.7 Hz, 6H), 6.54 (d, J=16 Hz, 3H), 7.54 (d, J=16 Hz, 3H), 7.67 (d, J=8 Hz, 6H), 7.84 (d, J=8 Hz, 6H), 10.73 (m, 3H).

UV: λₘₐₓ=358 nm; εₘₐₓ=154000 M⁻¹ cm⁻¹ (DMSO).

### Example 33

### Synthesis de 2,5,8-tris-(3-((E)-3-(2-ethylhexyloxy)-3-oxoprop-1-enyl)phenylamino)-1,3,4,6,7,9,9b-heptaazafenaleno

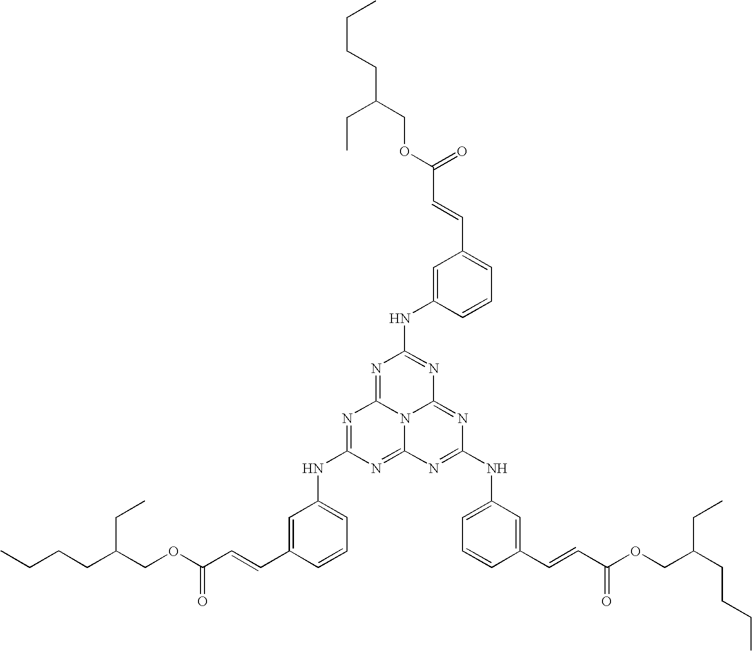

Following the method described in example 1. Yield: 73%.

M.P. 206-210° C.

¹H NMR (300 MHz, DMSO-d₆): δ 0.86 (m, 18H), 1.27 (m, 32H), 1.59 (m, 3H), 4.04 (m, 6H), 6.53 (d, J=16 Hz, 3H), 7.45 (m, 9H), 7.80 (m, 6H), 10.53 (m, 3H).

UV: λₘₐₓ=290 nm; εₘₐₓ=126000 M⁻¹ cm⁻¹ (DMSO).

### Example 34

### Synthesis of 2-(3-((E)-3-(2-ethylhexyloxy)-3-oxoprop-1-enyl)phenylamino)-5,8-bis-(4-((E) -3-(2-ethylhexyloxy)-3-oxoprop-1-enyl)phenylamino)-1,3,4,6,7,9,9b-heptaazaphenalene

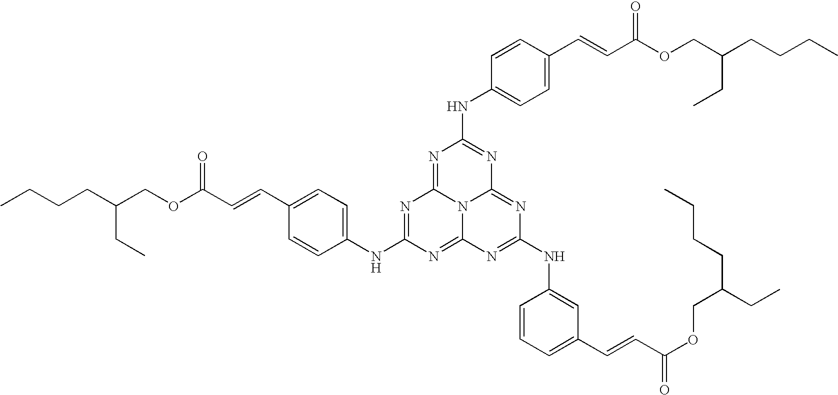

Following the method described in example 7.

¹H NMR (300 MHz, DMSO-d₆): δ 0.86 (m, 18H), 1.27 (m, 32H), 1.34 (m, 3H), 4.04 (m, 6H), 6.55 (m, 3H), 7.60 (m, 15H), 10.74 (m, 3H).

UV: λₘₐₓ=288 nm; εₘₐₓ=66000 M⁻¹ cm⁻¹; λₘₐₓ=354 nm; εₘₐₓ=113000 M⁻¹ cm⁻¹ (DMSO).

### Example 35

### Synthesis of 2,5,8-tris-(3-nitrophenylamino)-1,3,4,6,7,9,9b-heptaazaphenalene

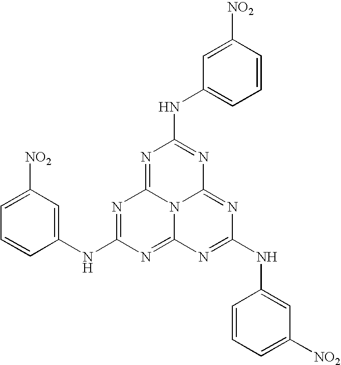

Following the method described in example 1. Yield: 76%.

¹H NMR (300 MHz, DMSO-d₆): δ 7.58 (t, J=8 Hz, 3H), 7.87 (d, J=7 Hz, 3H), 8.05 (d, J=7 Hz, 3H), 8.75 (s, 3H), 10.95 (m, 3H).

UV: λₘₐₓ=308 nm; εₘₐₓ=113000 M⁻¹ cm⁻¹ (DMSO).

### Example 36

### Synthesis of 2,5,8-tris-(4-(2-butyloctyloxycarbonyl)phenylamino)-1,3,4,6,7,9,9b-heptaazaphenalene

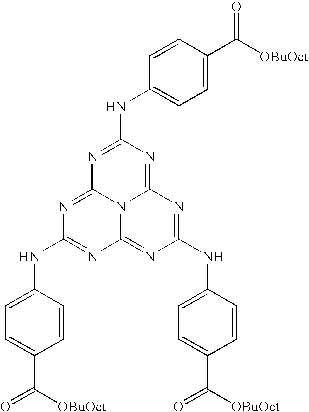

Following the method described in example 1. Yield: 85%. M.P. 215-222° C.

¹H NMR (300 MHz, DMSO-d₆): δ 0.88 (m, 18H), 1.28 (m, 48H), 1.76 (m, 3H), 4.21 (m, 6H), 7.70 (m, 6H), 7.99 (6H).

MS-EI (m/z): 1084 (M+1).

UV: λₘₐₓ=326 nm; εₘₐₓ=119000 M⁻¹ cm⁻¹ (CHCl₃).

### Example 37

### Synthesis of 2,5,8-tris-((3-(E)-styrylphenyl)amino)-1,3,4,6,7,9,9b-heptaazaphenalene

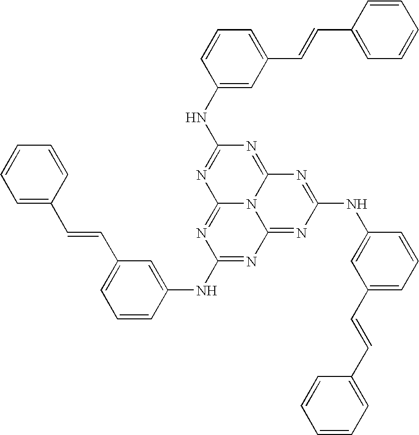

Following the method described in example 1. Yield: 57%.

¹H NMR (300 MHz, DMSO-d₆): δ 7.39 (m, 12H), 7.67 (m, 15H), 7.89 (m, 6H), 10.62 (m, 3H).

UV: λₘₐₓ=316 nm; εₘₐₓ=167000 M⁻¹ cm⁻¹ (DMSO).

### Example 38

### Synthesis of 2,5,8-tris-(4-(²-hexyldecyloxycarbonyl)phenylamino)-1,3,4,6,7,9,9b-heptaazaphenalene

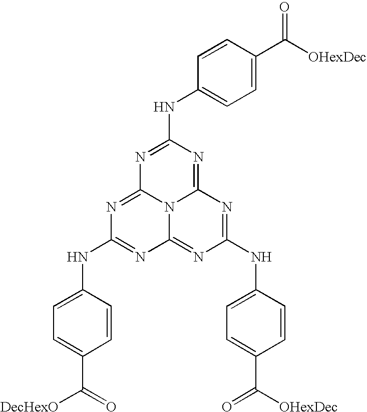

Following the method described in example 1. Yield: 87%.

M.P. 141-144° C.

¹H NMR (300 MHz, DMSO-d₆): δ 0.88 (m, 18H), 1.28 (m, 72H), 1.76 (m, 3H), 4.21 (m, 6H), 7.70 (m, 6H), 7.99 (m, 6H).

MS-EI (m/z): 1252 (M+1).

UV: λₘₐₓ=324 nm; εₘₐₓ=121000 M⁻¹ cm⁻¹ (DMSO).

### Example 39

### Synthesis of 2,5,8-tris-(4-((3,5,5-trimethylhexyloxy)carbonyl)phenylamino)-1,3,4,6,7,9,9b-heptaazaphenalene

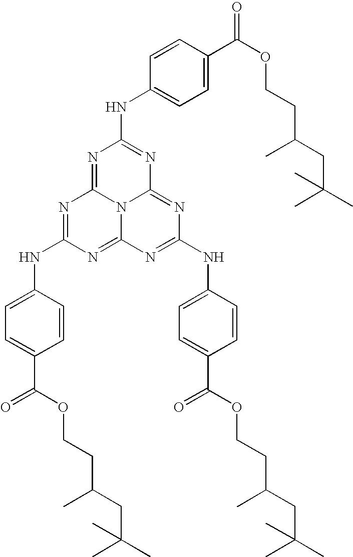

Following the method described in example 1. Yield: 86%.

¹H NMR (300 MHz, DMSO-d₆): δ 0.87 (s, 27H), 0.95 (d, J=6.3 Hz, 9H), 1.09 (m, 3H), 1.24 (m, 3H), 1.54 (m, 3H), 1.68 (m, 6H), 4.26 (m, 6H), 7.90 (m, 12H), 10.88 (s, 3H).

MS-EI (m/z): 957 (M+).

UV: λₘₐₓ=326 nm; εₘₐₓ=126000 M⁻¹ cm⁻¹ (DMSO).

### Example 40

### Synthesis of 2,5,8-tris-(4-(octadecyloxycarbonyl)phenylamino)-1,3,4,6,7,9,9b-heptaazaphenalene

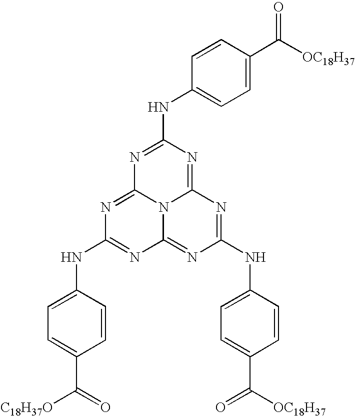

Following the method described in example 1. Yield: 61%.

¹H NMR (300 MHz, DMSO-d₆): δ 0.87 (t, J=6.8 Hz, 9H), 1.25 (m, 80H), 1.43 (bs, 10H), 1.76 (m, 6H), 4.30 (m, 6H), 7.68 (m, 6H), 8.03 (6H).

UV: λₘₐₓ=324 nm; εₘₐₓ=117000 M⁻¹ cm⁻¹ (DMSO).

### Example 41

### Synthesis of 2,5-bis-(3-(methoxy)phenylamino)-8-(2-(1-methyl-1H-pyrrol-2-yl)-1,3,4,6,7,9,9b-heptaazaphenalene

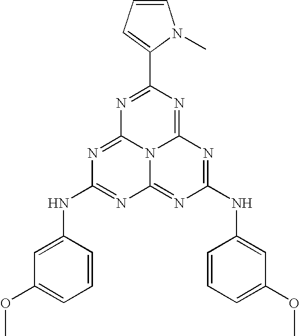

Following the method described in example 11. Yield: 47%.

¹H NMR (300 MHz, DMSO-d₆): δ 3.74 (s, 6H), 4.00 (s, 3H), 6.18 (m, 1H), 6.66 (m, 2H), 7.23 (m, 6H), 7.47 (s, 2H), 10.48 (s, 2H).

MS-EI (m/z): 494 (M+).

UV: λₘₐₓ=352 nm; εₘₐₓ=57000 M⁻¹ cm⁻¹ (DMSO).

### Example 42

### Synthesis of 2,5,8-tris-(4-(hexadecyloxycarbonyl)phenylamino)-1,3,4,6,7,9,9b-heptaazaphenalene

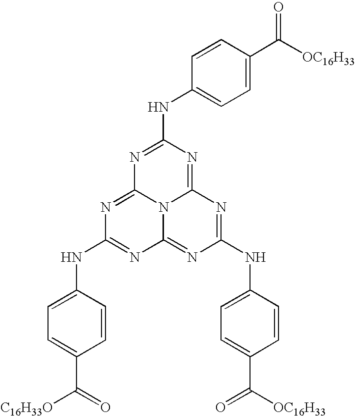

Following the method described in example 1. Yield: 91%.

M.P. 252-255° C.

¹H NMR (300 MHz, DMSO-d₆): δ 0.87 (t, J=6.8 Hz, 9H), 1.25 (m, 80H), 1.75 (m, 6H), 4.30 (m, 6H), 7.70 (m, 6H), 8.06 (m, 6H).

MS-EI (m/z): 1252 (M+1).

UV: λₘₐₓ=324 nm; εₘₐₓ=125000 M⁻¹ cm⁻¹ (DMSO).

### Example 43

### Synthesis of 2,5,8-tris-(4-((2-ethylhexyl)carbamoyl)phenylamino)-1,3,4,6,7,9,9b-heptaazaphenalene

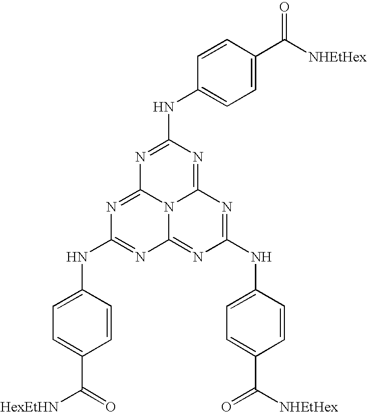

Following the method described on example 1. Yield: 50%.

¹H NMR (300 MHz, DMSO-d₆): δ 0.85 (m, 18H), 1.25 (m, 24H), 1.53 (m, 3H), 3.16 (m, 6H), 7.81 (m, 12H), 8.24 (m, 3H), 10.85 (m, 3H).

MS-EI (m/z): 912 (M+).

UV: λₘₐₓ=326 nm; εₘₐₓ=118000 M⁻¹ cm⁻¹ (CHCl₃).

### Example 44

### Synthesis of 2,5,8-tris-(4-(dodecyloxycarbonyl)phenylamino)-1,3,4,6,7,9,9b-heptaazaphenalene

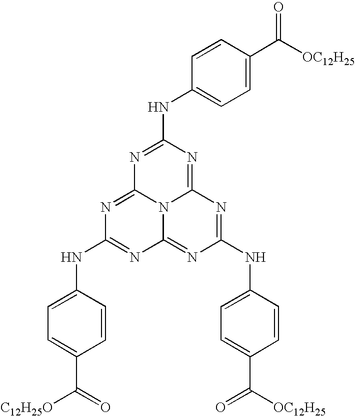

Following the method described in example 1. Yield: 87%.

¹H NMR (300 MHz, DMSO-d₆): δ 0.87 (t, J=6.8 Hz, 9H), 1.26 (m, 54H), 1.76 (m, 6H), 4.30 (m, 6H), 7.70 (m, 6H), 7.80 (bs, 3H), 8.05 (m, 6H).

UV: λₘₐₓ=326 nm; εₘₐₓ=87000 M⁻¹ cm⁻¹ (DMSO).

### Example 45

### Synthesis of 2,5,8-tris-(4-(1,3,3-trimethylbicyclo[2.2.1]heptan-2-yloxy)phenylamino)-1,3,4,6,7,9,9b-heptaazaphenalene

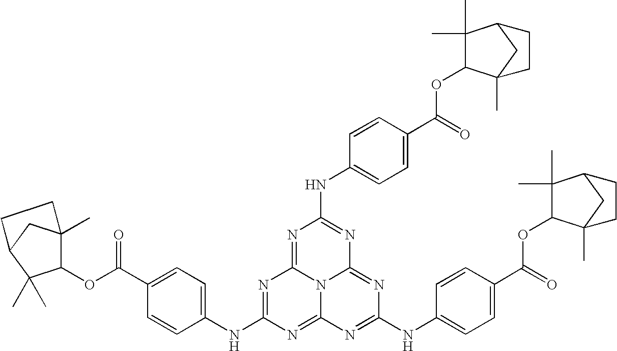

Following the method described in example 1. Yield: 89%.

¹H NMR (300 MHz, DMSO-d₆): δ 0.83 (s, 9H), 1.11 (s, 9H), 1.18 (s, 9H), 1.40-1.70 (m, 21H), 4.60 (s, 3H), 7.72 (m, 6H), 8.09 (m, 6H).

MS-EI (m/z): 988 (M+1).

UV: λₘₐₓ=330 nm; εₘₐₓ=150000 M⁻¹ cm⁻¹ (DMSO).

### Example 46

### Synthesis of 2,5,8-tris-(1H-indol-5-ylamino)-1,3,4,6,7,9,9b-heptaazaphenalene

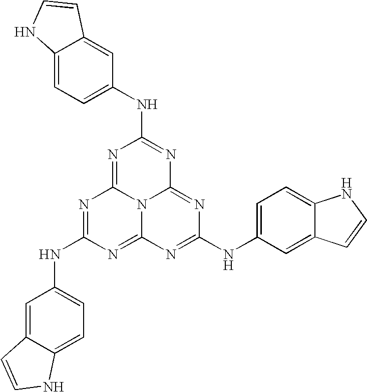

Following the method described in example 1. Yield: 53%.

¹H NMR (300 MHz, DMSO-d₆): δ 6.38 (m, 3H), 7.32 (m, 9H), 8.06 (m, 3H), 10.19 (s, 3H), 11.03 (s, 3H). MS-EI (m/z): 564 (M+1).

UV: λₘₐₓ=331 nm; εₘₐₓ=46000 M⁻¹ cm⁻¹ (DMSO).

### Example 47

### Synthesis of 2,5,8-tris-(4-((3,7-dimethyloctyloxy)carbonyl)phenylamino)-1,3,4,6,7,9,9b-heptaazaphenalene

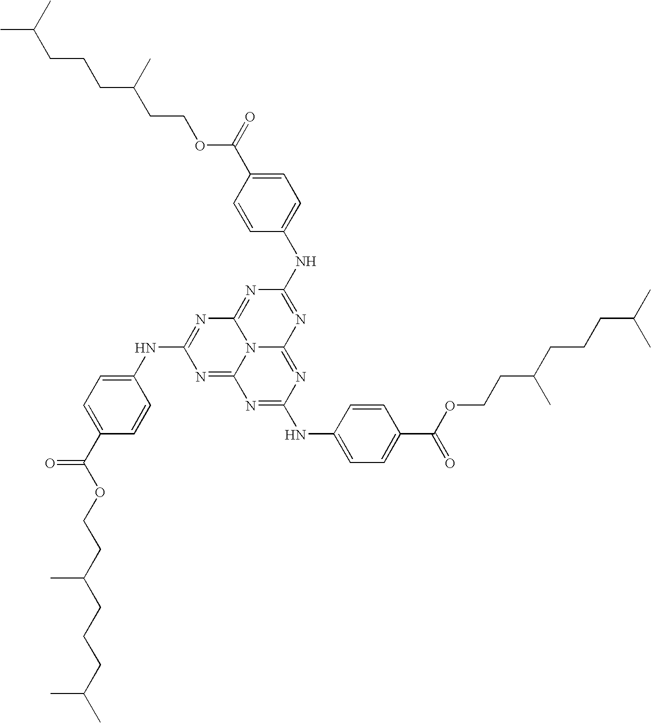

Following the method described in example 1. Yield: 74%.

¹H NMR (300 MHz, DMSO-d₆): δ 0.83 (d, J=6.6 Hz, 18H), 0.92 (d, J=6.4 Hz, 18H), 1.12-1.75 (m, 36H), 4.30 (m, 6H), 7.92 (bs, 12H), 10.90 (s, 3H).

UV: λₘₐₓ=329 nm; εₘₐₓ=125000 M⁻¹ cm⁻¹ (DMSO). Example 48

### Synthesis of 2,5,8-tris-(2-amino-4,5-dimethylphenylamino)-1,3,4,6,7,9,9b-heptaazaphenalene

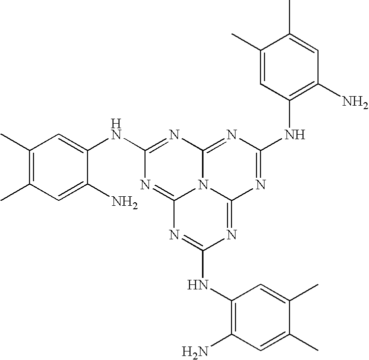

Following the method described in example 1. Yield: 77%.

¹H NMR (300 MHz, DMSO-d₆): δ 2.20 (s, 18H), 7.20 (m, 6H).

MS-EI (m/z): 576 (M+1).

UV: λₘₐₓ=270 nm; εₘₐₓ=44000 M⁻¹ cm⁻¹ (DMSO).

### Example 49

### Synthesis of 2,5,8-triphenyl-1,3,4,6,7,9,9b-heptaazaphenalene

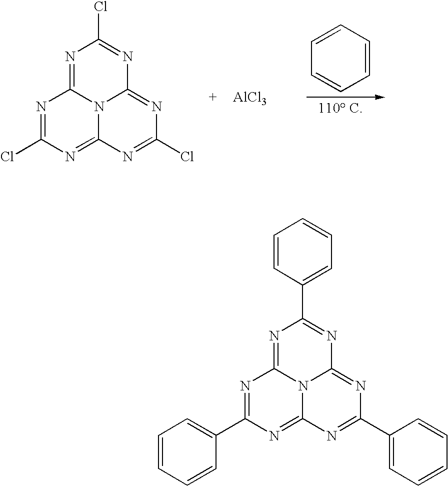

A mixture of 2,5,8-trichloro-1,3,4,6,7,9,9b-heptaazaphenalene (50 mg, 0.18 mmol) and aluminium trichloride (79 mg, 0.59 mmol) in benzene (2 mL) is heated at reflux. After 6 hours, HCl 1N (2 mL) is added and left at reflux for 30 minutes. The system is allowed to cool and the solid is filtered by porous plate and washed with HCl 6N (3×10 mL) and H₂O ((3×10 mL). This yields 2,5,8-triphenyl-1,3,4,6,7,9,9b-heptaazaphenalene.

MS-EI (m/z): 402 (M+1).

The following examples by way of non-restrictive illustration of the present inventions will lead to final products trough the methods of general knowledge by a person skilled in the art.

### Example 50

### Formulation in Oil

| | % w/w |
|---|---|
| Mineral Oil (Liquid Paraffin) | 59.85 |
| Arlamol HD (Uniqema) (Isohexadecane) | 16.00 |
| Arlamol S7 (Uniqema) (cyclomethicone, PPG-15, stearil ether | 16.00 |
| ParsolMCX (DSM) (ethylhexyl methoxycinnamate) | 5.00 |
| Perfume | 0.15 |
| 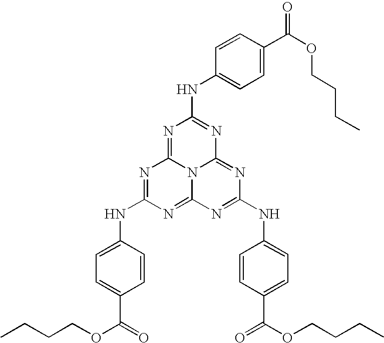 | 3.00 |

### Example 51

### Formulation in Form of Oil/Water Cream

| | % by weight |
|---|---|
| A) PEG-100 Stearate (Simulsol M59 (Seppic)) | 2.00 |
| Glyceryl Stearate (Cutina MS (Henkel)) | 1.00 |
| Cetearil Alcohol (Lanette or (Henkel)) | 2.50 |
| Stearic Acid | 5.00 |
| Propyleneglycol | 7.50 |
| Dicaprylate/dicaprate (Estol 1526 PDCC) | |
| Triglyceride (Myritol 318 (Henkel) | |
| Caprylic/capric | 3.00 |
| Dimethicone (SF 18-350 (General Electric) | 0.50 |
| Tocopheryl Acetate | 0.50 |
| 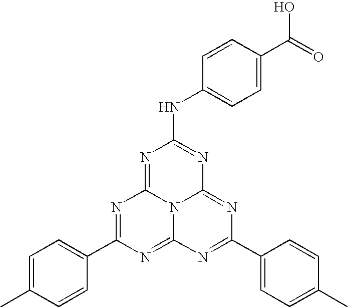 | 6.00 |
| B) Titanium Dioxide (and) | 4.00 |
| Aluminium Hydroxide (and) | |
| Stearic Acid (MT-T100 TV (Tayca)) | |
| Isohexadecane (Permethyl 101A (Presperse) | 5.00 |
| Cyclomethicone (SF 1204 (General Electric) | 2.50 |
| C) Water | up to 100 |
| Potassium Cethylphosphate (Amphisol K (Roche)) | 0.50 |
| D) PNC 30 (Sodium Acrylates/Cross-linked Polymer | 0.15 |
| Vinyl Isodecanoate) | |
| E) Butylenegylcol | 1.50 |
| ABIOL (Urea Imidazolidinil) | 0.30 |
| Methylparaben | 0.20 |
| Propylparaben | 0.10 |
| F. Perfume | 0.30 |

### Example 52

### Formulation in Form of Oil/Water Cream

| | % by weight |
|---|---|
| A) PEG-100 Stearate (Simulsol M59 (Seppic)) | 2.00 |
| Glyceryl Stearate (Cutina MS (Henkel)) | 1.00 |
| Cetearyl Alcohol (Lanette or (Henkel)) | 2.50 |
| Stearic Acid | 5.00 |
| Propyleneglycol | 7.50 |
| Dicaprylate/dicaprate (Estol 1526 PDCC) | |
| Triglyceride (Myritol 318 (Henkel) | |
| Caprylic/Capric | 3.00 |
| Dimethicone (SF 18-350 (General Electric) | 0.50 |
| Tocopheryl Acetate | 0.50 |
| 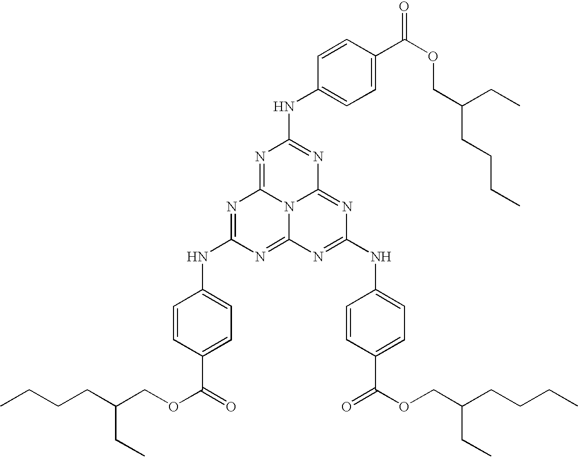 | 6.00 |
| B) Titanium Dioxide (and) | 4.00 |
| Aluminum Hydroxide (and) | |
| Stearic Acid (MT-T100 TV (Tayca)) | |
| Isohexadecane (Permethyl 101A (Presperse) | 5.00 |
| Cyclomethicone (SF 1204 (General Electric) | 2.50 |
| C) Water | up to 100 |
| Potassium Cethylphosphate (Amphisol K (Roche)) | 0.50 |
| D) PNC 30 (Sodium Acrylates/Cross-linked Polymer | 0.15 |
| Vinyl Isodecanoate) | |
| E) Butyleneglycol | 1.50 |
| ABIOL (Urea Imidazolidinyl) | 0.30 |
| Methylparaben | 0.20 |
| Propylparaben | 0.10 |
| F. Perfume | 0.30 |

1. A heptaazaphenalene derivative of general formula (I): 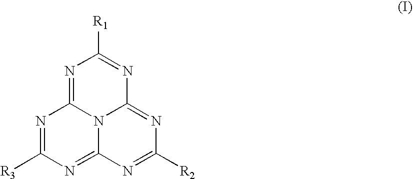
wherein
R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;
R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;
R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S;
and wherein the substituents of the radicals that are optionally substituted are selected from the group consisting of a linear or branched alkyl radical that contains from 1 to 8 carbon atoms; a C₃-C₆ cycloalkyl radical; C₂-C₆ alkenyl; C₂-C₆ alkenyl-COOR₇; C₂-C₆ alkenyl-aryl; C₁-C₈ alkoxide; aryl; saturated, unsaturated or aromatic heterocyclic group containing from 5 to 10 atoms that can contain 1, 2 or 3 heteroatoms selected from O, N and S; a —COOR₇ radical; a —CONR₈R₉ radical; a —COR₁₀ radical; an hydroxyl radical; an NR₈R₉ radical; a sulfur-containing radical; a nitro radical; an halogen such as chlorine or fluorine; a C₁-C₈ alkoxide; an optionally substituted linear or branched alkyl chain radical having from 1 to 6 carbon atoms, wherein the alkoxide or the alkyl radical can be substituted by at least one hydroxyl group an —SO₃M radical a —N(R₁₁)₂ radical, an —N(R₁₁)₃⁺ radical or a group of general formula (II): 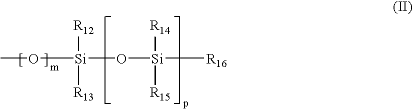
where
m=0 or 1;
p=0, 1, 2, 3 or 4
R₁₂, R₁₃, R₁₄, R₁₅ and R₁₆ are the same as or different from each other and represent an optionally substituted alkyl radical having from 1 to 6 carbon atoms an alkoxide radical having from 1 to 6 carbon atoms, an optionally substituted aryl radical or an —OSi(R₁₇)₃ radical;
R₁₇ represents an alkyl radical having from 1 to 6 carbon atoms, an alkoxide radical having from 1 to 6 carbon atoms or an optionally substituted aryl radical;
M is H, Na or K;
R₇, R₈ and R₉ are independently selected from hydrogen; an optionally substituted linear or branched alkyl radical having from 1 to 18 carbon atoms; a substituted or unsubstituted ary radical; a saturated, unsaturated or aromatic heterocycle having from 5 to 10 atoms that can contain 1, 2 or 3 heteroatoms selected from O, N and S; a C₃-C₁₂ cycloalkyl radical; or
R⁸ and R⁹ can be fused, forming together with the nitrogen a mono- or polycyclic saturated, unsaturated or aromatic ring system having from 5 to 10 atoms that can optionally contain 1 or 2 heteroatoms selected from O, N and S;
R₁₀ is an optionally substituted alkyl radical, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;
R₁₁ is an optionally substituted alkyl radical;
any of the above mentioned groups could also be optionally substituted in at least one position;
or a pharmaceutically, dermatologically or cosmetically acceptable salt, tautomer, isomer or solvate thereof,
with the condition that if R₁, R₂ and R₃ are identical they may not be an unsubstituted phenyl; and
with the condition that if R₁, R₂ and R₃ are the same and are OR₄, then R4 is not hydrogen; and
with the condition that if R₁, R₂ and R₃ are the same and are OR₄, then OR4 is not n-butyl, ethyl, phenyl, benzyl, 2,6-dimethylphenyl, 3,5-dimethylphenyl, 2,2,3,3,4,4,4-heptafluorobutyl, 2,2,3,3,3-pentafluoropropyl, 2,2,2-trifluoroethyl and hydroxymethyl; and
with the condition that when R₁, R₂ and R₃ are the same and are OR₄ if R₄ is ethyl for two of the radicals R₁, R₂ and R₃, then R₄ is not hydrogen for the third radical R₁, R₂ and R₃; and
with the condition that when R₁, R₂ and R₃ are the same and are NR₅R₆, then R₅ and R₆, if identical, are not hydrogen, ethyl, n-butyl, benzyl, n-heptyl, unsubstituted phenyl, cyclohexyl, 2-pyridyl, or hydroxymethyl; and
with the condition that when R₁, R₂ and R₃ are the same and are NR₅R₆, if one of R₅ or R₆ is hydrogen, the other radical R₅ or R₆ is not n-butyl, unsubstituted phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl; and
with the condition that when R₁, R₂ and R₃ are the same and are NR₅R₆, if one of R₅ or R₆ is phenyl, the other R₅ or R₆ radical is not methyl; and
with the condition that when R₁, R₂ and R₃ are NR₅R₆ if R₅ and R₆ are the same for two of the radicals R₁, R₂, R₃, and represent n-heptyl, and for the third radical of R₁, R₂, R₃, being NR₅R₆, R₅ or R₆ is phenyl, then the other of R₅ or R6 for the third radical is not phenyl; and
with the condition that when R₁, R₂ and R₃ are NR₅R₆ if R₅ and R₆ are the same for two of the radicals R₁, R₂, R₃, and represent phenyl, and for the third radical of R₁, R₂, R₃, being NR₅R₆, R₅ or R₆ is n-heptyl, then the other R₅ or R₆ for the third radical is not n-heptyl;
with the condition that if R₁, R₂ and R₃ are identical they may not be a phenyl, 2,4,6-trimethylphenyl, 2,3,5,6-tetramethylphenyl, x-methylphenyl, x,x′-dimethylphenyl, x,x′,x″-trimethylphenyl, x-ethylphenyl, or x,x′-diethylphenyl, x,x′,x″-triethylphenyl.

2. A heptaazaphenalene derivative according to claim 1, having one of the following general formulae: 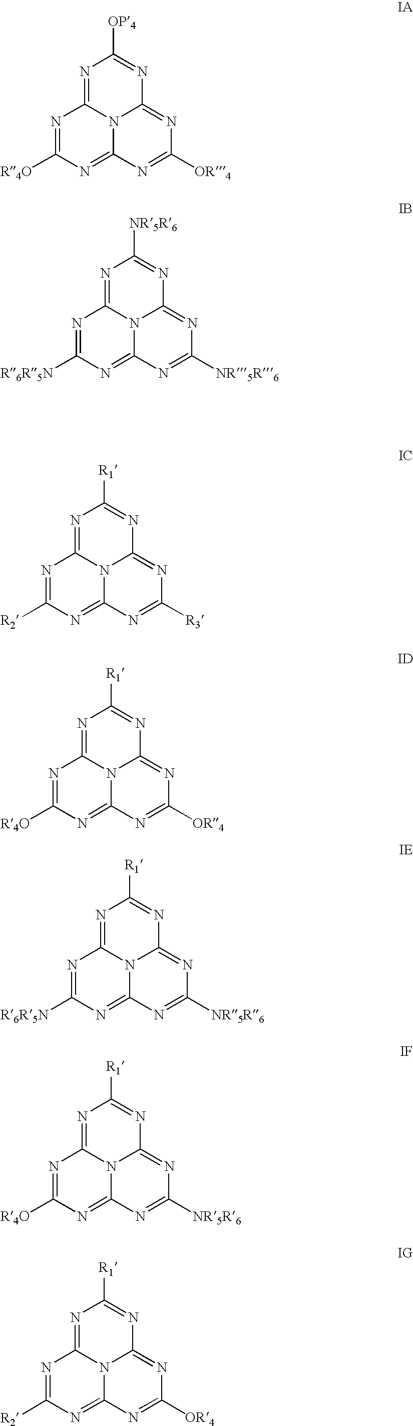 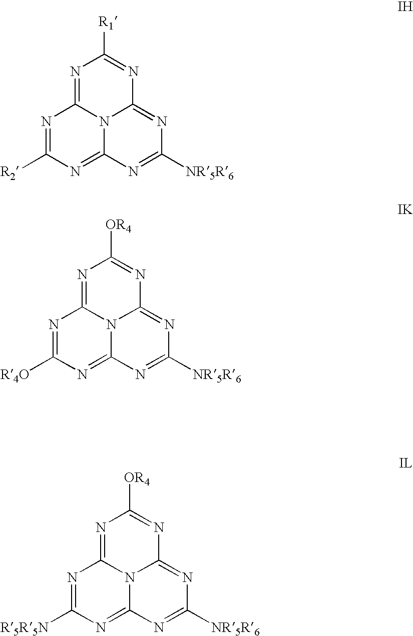
wherein R′₁, R′₂ and R′₃ are the same as or different from each other and represent an optionally substituted mono- or polycyclic aryl radical; an optionally substituted saturated, unsaturated or aromatic heterocyclic radical having from 5 to 10 atoms that can optionally contain 1, 2 or 3 heteroatoms selected from O, N and S;
R′₄, R″₄ and R′″₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;
R′₅, R″₅, R′″₅, R′₆, R″₆ and R′″₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or the respective R′₅, R′₆; R″₅, R″₆ and R′″₅, R′″₆ pairs are fused to form together with the nitrogen a saturated, unsaturated or aromatic mono- or polycyclic ring system having from 4 to 10 atoms that can optionally contain 1 or 2 heteroatoms selected from N, O and S;

3. A heptaazaphenalene derivative according to claim 2 having the general formula (IA): 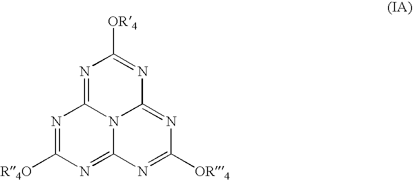
where each of R′₄, R″₄ and R′″₄ represent a cycloalkyl radical having from 3 to 12 carbon atoms; an optionally substituted mono- or polycyclic aryl radical; an optionally substituted saturated, unsaturated or aromatic heterocyclic radical containing from 5 to 14 atoms that can optionally contain 1, 2 or 3 heteroatoms selected from O, N and S;
with the condition that when R′₄, R″₄ and R′″₄ are the same, they are not phenyl, benzyl, 2,6-dimethylphenyl or 3,5-dimethylphenyl.

4. A heptaazaphenalene derivative according to claim 2, wherein each of R₄, R′₄, R″₄ and R′″₄ are selected from an aryl group that can be substituted in at least one position, with the substituent being an optionally substituted C₃-C₁₂ cycloalkyl radical; an optionally substituted C₂-C₆ alkenyl radical; an optionally substituted aryl; an optionally substituted saturated, unsaturated or aromatic heterocycle having from 5 to 10 atoms that can contain 1, 2 or 3 heteroatoms selected from O, N and S; a —COOR₇ radical; a —CONR₈R₉ radical; a —COR₁₀ radical; an hydroxyl radical; an halogen such as chlorine or fluorine; C₁-C₈ alkoxide; an optionally substituted linear or branched alkyl chain radical having from 1 to 6 carbon atoms, wherein the alkoxide or the alkyl radical can be substituted by at least one hydroxyl group, an —SO₃M radical, a —N(R₁₁)₂ radical, an —N(R₁₁)₃⁺ radical, or a group of general formula (II): 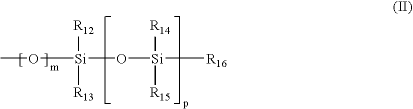
where
m=0 or 1;
p=0, 1, 2, 3 or 4
R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or an —OSi(R₁₇)₃ radical;
R₁₇ represents an optionally substituted alkyl radical having from 1 to 6 carbon atoms, an alkoxide radical having from 1 to 6 carbon atoms or an optionally substituted aryl radical;
M is H, Na or K;
R₇, R₈ and R₉ are independently selected from hydrogen; an optionally substituted aryl radical; an optionally substituted linear or branched alkyl radical having from 1 to 18 carbon atoms; an optionally substituted saturated, unsaturated or aromatic heterocycle having from 5 to 10 atoms that can contain 1, 2 or 3 heteroatoms selected from O, N and S; a C₃-C₁₂ cycloalkyl radical; or
R₈ and R₉ can be fused, forming together with the nitrogen a mono- or polycyclic saturated, unsaturated or aromatic ring system having from 5 to 10 atoms that can optionally contain 1 or 2 heteroatoms selected from O, N and S;
R₁₀ is an optionally substituted alkyl radical, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;
R₁₁ is an optionally substituted alkyl radical,
with the condition that when R₁, R₂ and R₃ are the same, R₄ is not phenyl, benzyl, 2,6-dimethylphenyl or 3,5-dimethylphenyl.

5. A heptaazaphenalene derivative according to claim 2 having the general formula (IB): 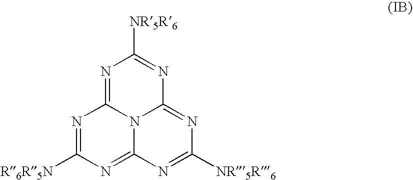
wherein the radicals within each radical pair R′₅R′₆, R″₅R″₆, and R′″₅R′″₆ are different from each other and represent hydrogen; an optionally substituted linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can optionally contain 1, 2 or 3 heteroatoms selected from O, N and S;
or the radical pairs R′₅R′₆, R″₅R″₆, or R′″₅R′″₆ are fused and form together with the nitrogen a saturated, unsaturated or aromatic mono- or polycyclic ring system having from 4 to 10 atoms that can optionally contain 1 or 2 heteroatoms selected from N, O and S;
with the condition that if one of the radicals within each radical pair R′₅R′₆, R″₅R″₆, and R′″₅R′″₆ is hydrogen, the other radical is not n-butyl, phenyl, hydroxymethyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl;
with the condition that if one of the radicals within each radical pair R′₅R′₆, R″₅R″₆, and R′″₅R′″₆ is phenyl, the other radical is different from not methyl.

6. A heptaazaphenalene derivative according to claim 2, wherein one radical of the pair R₅R₆ or optionally one radical of the pairs R′₅R′₆, R″₅R″₆ or R′″₅R′″₆ represents an aryl group that can be substituted in at least one position, with said substituent being a C₃-C₁₂ cycloalkyl radical; an optionally substituted C₂-C₆ alkenyl radical; an optionally substituted aryl radical; an optionally substituted saturated, unsaturated or aromatic heterocycle having from 5 to 10 atoms that can contain 1, 2 or 3 heteroatoms selected from O, N and S; a —COOR₇ radical; a —CONR₈R₉ radical; a —COR₁₀ radical; an hydroxyl radical; an halogen such as chlorine or fluorine; C₁-C₈ alkoxide; an optionally substituted linear or branched alkyl radical having from 1 to 6 carbon atoms, where the alkoxide radical or the alkyl radical can be optionally substituted by at least one —SO₃M group, an —N(R₁₁)₂ radical, an —N(R₁₁)₃⁺ radical, or group of general formula (II): 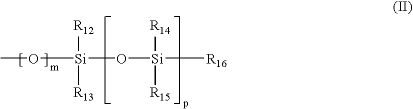
where
m=0 or 1;
p=0, 1, 2, 3 or 4
R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or a —OSi(R₁₇)₃ radical;
R₁₇ represents an alkyl radical having from 1 to 6 carbon atoms, an alkoxide radical having from 1 to 6 carbon atoms or an optionally substituted aryl radical;
M is H, Na or K;
R₇, R₈ and R₉ are independently selected from hydrogen; an optionally substituted aryl radical; an optionally substituted linear or branched alkyl radical having from 1 to 18 carbon atoms; an optionally substituted saturated, unsaturated or aromatic heterocycle having from 5 to 10 atoms that can contain 1, 2 or 3 heteroatoms selected from O, N and S; a C₃-C₁₂ cycloalkyl radical; or
R₈ and R₉ can be fused to form together with the nitrogen a mono- or polycyclic saturated, unsaturated or aromatic ring system having from 5 to 10 atoms that can optionally contain 1 or 2 heteroatoms selected from O, N and S;
R₁₀ is an optionally substituted saturated or unsaturated, linear or branched alkyl radical having from 1 to 6 carbon atoms, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;
R₁₁ is an optionally substituted alkyl radical;
with the condition that if one radical of the pair R₅R₆ or optionally one radical of the pairs R′₅R′₆, R″₅R″₆ or R′″₅R′″₆ is an unsubstituted phenyl, 4-methoxy-9,10-dihydro-9,10-dioxoanthracene-1-yl, 9,10-dihydro-9,10-dioxoanthracene-1-yl, 9,10-dihydro-9,10-dioxoanthracene-2-yl, or 5-benzoylamino-9,10-dihydro-9,10-dioxoanthracene-1-yl, the other radical is not hydrogen.

7. (canceled)

8. A heptaazaphenalene derivative according to claim 1, in which R₁, R₂ and R₃, are the same as or different from each other and represent naphthyl, pyrrole, thiophene, indol, pyrazol, imidazol, triazol, benzothiophene, benzimidazole, benzopyrazole, oxazole, isoxazole, benzofuran, all of them optionally substituted, or else a radical of general formula (III) 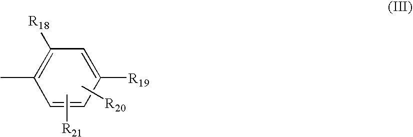
wherein
R₁₈ represents a hydrogen; or a hydroxyl radical; an —OR₂₂ radical; an optionally substituted saturated or unsaturated linear or branched alkyl radical that contains from 1 to 18 carbon atoms; or an optionally substituted linear or branched chain C₁-C₁₈ alkoxyde radical;
R₁₉ represents a hydrogen; a hydroxyl radical; an optionally substituted aryl radical; an optionally substituted saturated, unsaturated or aromatic heterocyclic radical having from 5 to 10 atoms that can optionally contain 1, 2 or 3 heteroatoms selected from O, N and S; a —COOR₇ radical; a —CONR₈R₉ radical; an —OR₂₂ radical; an optionally substituted —COR₁₀ radical; a C₃-C₆ cycloalkyl radical; an optionally substituted, saturated or unsaturated linear or branched alkyl radical that contains from 1 to 18 carbon atoms, optionally substituted by at least one hydroxyl radical, an —SO₃M, —N(R₁₁)₂ or —N(R₁₁)₃⁺ group, or else by a group of general formula (II): 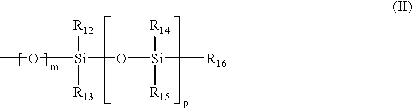
wherein
m=0 or 1;
p=0, 1, 2, 3 or 4;
R₁₂, R₁₃, R₁₄, R₁₅ and R₁₆ are the same as or different from each other and represent an optionally substituted alkyl radical having from 1 to 6 carbon atoms, an alkoxide radical having from 1 to 6 carbon atoms, an optionally substituted aryl radical or a —OSi(R₁₇)₃ radical;
R₁₇ represents an alkyl radical having from 1 to 6 carbon atoms, an alkoxide radical having from 1 to 6 carbon atoms or an optionally substituted aryl radical;
M is H, Na or K;
R₇, R₈ and R₉ are independently selected from hydrogen; an optionally substituted aryl radical; an optionally substituted linear or branched alkyl radical having from 1 to 18 carbon atoms; an optionally substituted saturated, unsaturated or aromatic heterocycle having from 5 to 10 atoms that can contain 1, 2 or 3 heteroatoms selected from O, N and S; a C₃-C₁₂ cycloalkyl radical; or
R₈ and R₉ can be fused to form together with the nitrogen a mono- or polycyclic saturated, unsaturated or aromatic ring system having from 5 to 10 atoms that can optionally contain 1 or 2 heteroatoms selected from O, N and S;
R₁₀ is an optionally substituted saturated or unsaturated, linear or branched alkyl radical having from 1 to 6 carbon atoms, or an optionally substituted aryl radical, or R₁₀ is fused to form a mono- or polycyclic saturated, unsaturated or aromatic ring system having from 5 to 10 atoms that can optionally contain 1 or 2 heteroatoms selected from O, N and S;
R₁₁ is an optionally substituted alkyl radical;
R₂₂ represents an optionally substituted aryl radical; an optionally substituted saturated, unsaturated or aromatic heterocyclic radical having from 5 to 10 atoms that can optionally contain 1, 2 or 3 heteroatoms selected from O, N and S; an optionally substituted —COR₁₀ radical; a C₃-C₁₂ cycloalkyl radical; a saturated or unsaturated linear or branched alkyl radical that contains from 1 to 18 carbon atoms, optionally substituted at least by one hydroxyl radical, a —SO₃M, —N(R₁₁)₂ or —N(R₁₁)₃⁺ group or else by a group of general formula (II): 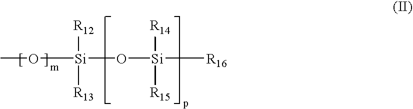
wherein
m=0 or 1;
p=0, 1, 2, 3 or 4
where R₇, R₈, R₉, R₁₀, R₁₁, R₁₂, R₁₃, R₁₄, R₁₅, R₁₆ and M are as defined above;
R₂₀ and R₂₁ can be the same or different and represent hydrogen; an optionally substituted saturated or unsaturated linear or branched alkyl radical that contains from 1 to 6 carbon atoms; an optionally substituted C₁-C₆ alkoxide radical; or an —SO₃M radical, where M is as defined above.

9. A derivative according to claim 1, in which R₅, and R₆, are different from each other and represent hydrogen, cyclopropyl, cyclohexyl, cyclobutyl, cycloheptyl, cyclopentyl, 4-(hydroxycarbonyl)phenyl, 4-(butoxycarbonyl)phenyl, 4-(2-ethylhexyloxycarbonyl)phenyl, 4-(2-butyloctyloxycarbonyl)phenyl, 4-(2-hexyldecyloxycarbonyl)phenyl, 4-(3,3,5-trimethylcyclohexyloxycarbonyl)phenyl, 4-(3,3,5-trimethylhexyloxycarbonyl)phenyl, 4-(octadecyloxycarbonyl)phenyl, 4-(hexadecyloxycarbonyl)phenyl, 4-(docecyloxycarbonyl)phenyl, 4-((2-ethylhexyl)carbamoyl)phenyl, 4-(L-menthyloxycarbonyl)phenyl, 4-styrylphenyl, 3-styrylphenyl, 3-((E)-3-(2-ethylhexyloxy)-3-oxoprop-1-enyl)phenyl, 4-((E)-3-(2-ethylhexyloxy)-3-oxoprop-1-enyl)phenyl, 4-methoxyphenyl, 3-methoxyphenyl, 3-nitrophenyl, phenyl, biphenyl-4-yl, 4-(imidazo[1,2-a]pyrridin-2-yl)phenyl, 4-(4,5,6,7-tetrahydro-2,6,6-trimethyl-4-oxoindol-1-yl)phenyl, 4-(1H-benzo[d]imidazol-2-yl)phenyl, hydroxymethyl, pyridine, heptyl, butyl, ethyl, 2-ethylhexyl, 4-(1,3,3-trimethylbicyclo[2.2.1]heptan-2-yloxy)phenyl, 1H-indol-5-yl, 4-((3,7-dimethyloctyloxy)carbonyl)phenyl, 2-amino-4,5-dimethylphenyl or n-propyl.

10. A derivative according to claim 1, in which R₄ represents 4-methoxyphenyl, naphthyl, cyclopentyl, cyclohexyl.

11. A derivative according to claim 1, in which R₇ represents hydrogen, methyl, ethyl, n-propyl, isopropyl, n-butyl, tert-butyl, n-pentyl, n-hexyl, 2-ethylhexyl, L-menthyl, 3,3,5-trimethylcyclohexanyl, 3,3,5-trimethylhexanyl, dodecyl, 2-butyloctyl, 2-hexyldecyl, hexadecyl, octadecyl, 3,7-dimethyloctyl, 1,3,3-trimethylbicyclo[2.2.1]heptan-2-yl, optionally substituted benzyl radical or an optionally substituted phenyl radical.

12. A heptaazaphenalene derivative according to claim 1, wherein R₈ and R₉, are the same or different, represent hydrogen, methyl, ethyl, n-propyl, isopropyl, n-butyl, tert-butyl, n-pentyl, n-hexyl, 2-ethylhexyl, L-menthyl, 3,3,5-trimethylcyclohexanyl, 3,3,5-trimethylhexanyl, dodecyl, hexadecyl, 2-butyloctyl, 2-hexyldecyl, octadecyl, 3,7-dimethyloctyl, 1,3,3-trimethylbicyclo[2.2.1]heptan-2-yl, optionally substituted benzyl radical or an optionally substituted phenyl radical.

13. A heptaazaphenalene derivative according to claim 1, wherein R₁₀ represents methyl, ethyl, n-propyl, n-butyl, tert-butyl or phenyl.

14. A derivative according to claim 1, wherein R₁₂, R₁₃, R₁₄, R₁₅ and R₁₆ represent methyl, ethyl, methoxy, ethoxy or phenyl.

15. A derivative according to claim 1, wherein R₁₇ represents methyl, ethyl, methoxy, ethoxy or phenyl.

16. A derivative according to claim 8, wherein R₁₈ represents a hydrogen, a hydroxyl radical, a methyl radical, a methoxy radical or an acyloxy radical.

17. A derivative according to claim 8, wherein R₁₉ represents a hydrogen, a hydroxyl radical, an acyloxy radical, a linear or branched chain, saturated or unsaturated alkoxide radical such as methoxy, ethoxy, n-propoxy, iso-propoxy, n-butoxy, tert-butoxy, 2-ethylhexyloxy, phenoxide, optionally substituted by at least one —SO₃M or —N(R₁₁)₃⁺ group.

18. A derivative according to claim 8, wherein R₂₀ and R₂₁ are the same as or different from each other and represent a hydrogen, a hydroxyl group, methoxy, ethoxy, n-propoxy, iso-propoxy, n-butoxy, tert-butoxy, n-pentoxy, hexyloxy or 2-ethylhexyloxy radical, optionally substituted by at least one —SO₃M group.

19. A derivative according to any claim 1, selected from the group consisting of:
2,5,8-tris-(4-(butoxycarbonyl)phenylamino)-1,3,4,6,7,9,9b-heptaazaphenalene;
2,5,8-tris-(4-(2-ethylhexyloxycarbonyl)phenylamino)-1,3,4,6,7,9,9b-heptaazaphenalene;
2,5,8-tris-(4-(imidazo[1,2-a]pyrridin-2-yl)-phenylamino) -1,3,4,6,7,9,9b-heptaazaphenalene;
2,5,8-tris-(4-(4,5,6,7-tetrahydro-2,6,6-trimethyl-4-oxo indol-1-yl)phenylamine)-1,3,4,6,7,9,9b-heptaazaphenalene;
2,5,8-tris-(biphenyl-4-ylamino)-1,3,4,6,7,9,9b-heptaazaphenalene;
2,5,8-tris-(4-(1H-benzo[d]imidazol-2-yl)phenylamino)-1,3,4,6,7,9,9b-heptaazaphenalene;
2,5-bis-(biphenyl-4-ylamino)-8-(4-(butoxycarbonyl)phenylamino)-1,3,4,6,7,9,9b-heptaazaphenalene;
2-(biphenyl-4-ylamino)-5-(4-(butoxycarbonyl)phenylamino)-8-(4-(2-ethylhexyloxycarbonyl)phenylamino)-1,3,4,6,7,9,9b-heptaazaphenalene;
2,5,8-tris-(2,4-dihydroxyphenyl)-1,3,4,6,7,9,9b-heptaazaphenalene;
2,5-dichloro-8-(1-methyl-1H-pyrrol-2-yl)-1,3,4,6,7,9,9b-heptaazaphenalene;
2,5-bis-(4-(butoxycarbonyl)phenylamino)-8-(2-(1-methyl-1H-pyrrol-2-yl)-1,3,4,6,7,9,9b-heptaazaphenalene;
2-(4-(carboxy)phenylamino)-5,8-bis-(4-methylphenyl)-1,3,4,6,7,9,9b-heptaazaphenalene;
2,5,8-tris-(4-(4,5,6,7-tetrahydro-2,6,6-trimethyl-4-oxoindol-1-yl)phenylamine)-1,3,4,6,7,9,9b-heptaazaphenalene;
2,5-bis-(4-(butoxycarbonyl)phenylamino)-8-(1-methyl-1H-pyrrol-2-yl)-1,3,4,6,7,9,9b-heptaazaphenalene;
2,5-bis(2,4-dihydroxyphenyl)-8-(1-methyl-1H-pyrazol-5-yl)-1,3,4,6,7,9,9b-heptaazaphenalene;
2,5-bis{[4-(2-ethylhexyloxy)-2-hydroxy]phenyl}-6-(1-methyl-1H-pyrazol-5 -yl)-1,3,4,6,7,9,9b-heptaazaphenalene;
2-[2,4-bis(2-ethylhexyloxy)phenyl]-5-[4-(2-ethylhexyloxy)-2-hydroxyphenyl]-8-(1-methyl-1H-pyrazol-5-yl)-1,3,4,6,7,9,9b-heptaazaphenalene;
2,5-bis(2,4-dihydroxyphenyl)-8-(1-phenyl-1H-pyrazol-5-yl)-1,3,4,6,7,9,9b-heptaazaphenalene;
2,5-bis{[4-(2-ethylhexyloxy)-2-hydroxy]phenyl}-8-(1-phenyl-1H-pyrazol-5-yl)-1,3,4,6,7,9,9b-heptaazaphenalene;
2,5-bis[4-(butoxycarbonyl)phenylamino]-8-(1-methyl-1H-pyrazol-5-yl)-1,3,4,6,7,9,9b-heptaazaphenalene;
2,5-bis{4-[(2-(ethylhexyloxy)carbonyl]phenylamino}-8-(1-methyl-1H-pyrazol-5-yl)-1,3,4,6,7,9,9b-heptaazaphenalene;
2,5-bis[4-(butoxycarbonyl)phenylamino]-8-(1-phenyl-1H-pyrazol-5-yl)-1,3,4,6,7,9,9b-heptaazaphenalene;
2,5-bis{4-[(2-ethylhexyloxy)carbonyl]phenylamino}-8-(1-phenyl-1H-pyrazol-5-yl)-1,3,4,6,7,9,9b-heptaazaphenalene;
2-(1-benzyl-1H-pyrrol-2-yl)-5,8-bis(2,4-dihydroxyphenyl)-1,3,4,6,7,9,9b-heptaazaphenalene;
2-(1-benzyl-1H-pyrrol-2-yl)-5,8-bis[4-(2-ethylhexyloxy)-2-hydroxyphenyl]-1,3,4,6,7,9,9b-heptaazaphenalene;
2-(1-benzyl-1H-pyrrol-2-yl)-5,8-bis[4-(butoxycarbonyl)phenylamino]-1,3,4,6,7,9,9b-heptaazaphenalene;
2-(1-benzyl-1H-pyrrol-2-yl)-5,8-bis(biphenyl-4-ylamino)-1,3,4,6,7,9,9b-heptaazaphenalene;
2-(1-benzyl-1H-pyrrol-2-yl)-5,8-bis(4-benzoylphenylamino)-1,3,4,6,7,9,9b-heptaazaphenalene;
2-(1-benzyl-1H-pyrrol-2-yl)-5,8-bis(9-oxo-9H-fluoren-3-ylamino)-1,3,4,6,7,9,9b-heptaazaphenalene;
2-(4-(tert-butylcarbamoyl)phenylamino)-5,8-bis-(4-(2-ethylhexyloxycarbonyl)phenylamino)-1,3,4,6,7,9,9b-heptaazaphenalene;
2,5-bis-[(4-(2-ethylhexyloxy)-2-hydroxy)-phenyl]-8-(4-methoxyphenyl)-1,3,4,6,7,9,9b-heptaazaphenalene;
2-(2-ethylhexylamino)-5,8-bis-(4-(5-(1,1-dimethylpropyl)benzo[d]oxazol-2-yl)phenylamino)-1,3,4,6,7,9,9b-heptaazaphenalene;
2,5,8-tris-(4-(1H-pyrazol-1-yl)phenylamino)-1,3,4,6,7,9,9b-heptaazaphenalene;
2,5,8-tris-(4-benzoylphenylamino)-1,3,4,6,7,9,9b-heptaazaphenalene;
2,5,8-tris-(4-butoxy-2-hydroxyphenyl)-1,3,4,6,7,9,9b-heptaazaphenalene;
2,5,8-tris-(naphthalen-2-ylamino)-1,3,4,6,7,9,9b-heptaazaphenalene;
2,5-bis-(4-(butoxycarbonyl)phenylamino)-8-(2-(1-methyl-1H-indol-3-yl)-1,3,4,6,7,9,9b-heptaazaphenalene;
2,5,8-tris-(biphenyl-4-yloxy)-1,3,4,6,7,9,9b-heptaazaphenalene;
2,5,8-tris-(3-methoxyphenylamino)-1,3,4,6,7,9,9b-heptaazaphenalene;
2,5,8-tris-(4-methoxyphenylamino)-1,3,4,6,7,9,9b-heptaazaphenalene;
2,5,8-tris-(4-((E)-3-ethoxy-3-oxoprop-1-enyl)phenylamino)-1,3,4,6,7,9,9b-heptaazaphenalene;
2,5,8-tris-(methoxycarbonyl-4′-biphenyl-4-ylamino)-1,3,4,6,7,9,9b-heptaazaphenalene;
2,5,8-tris-(4-(methoxycarbonyl)phenylamino)-1,3,4,6,7,9,9b-heptaazaphenalene;
2,5,8-tris-(4-(1H-benzo[d]imidazol-2-yl)-3-hydroxyphenylamino)-1,3,4,6,7,9,9b-heptaazaphenalene;
2,5,8-tris-(4-(phenylamino)phenylamino)-1,3,4,6,7,9,9b-heptaazaphenalene;
2,5,8-tris-((4-(E)-styrylphenyl)amino)-1,3,4,6,7,9,9b-heptaazaphenalene;
2,5,8-tris-(2-ethylhexylamino)-1,3,4,6,7,9,9b-heptaazaphenalene;
2,5,8-tris-(4-(L-menthylcarbonyl)phenylamino)-1,3,4,6,7,9,9b-heptaazaphenalene;
2,5,8-tris-(4-((3,3,5-trimethylcyclohexyloxy)carbonyl)phenylamino)-1,3,4,6,7,9,9b-heptaazaphenalene;
2,5,8-tris-(4-((E)-3-(2-ethylhexyloxy)-3-oxoprop-1-enyl)phenylamino)-1,3,4,6,7,9,9b-heptaazaphenalene;
2-(3-((E)-3-(2-ethylhexyloxy)-3-oxoprop-1-enyl)phenylamino)-5,8-bis-(4-((E)-3-(2-ethylhexyloxy)-3-oxoprop-1-enyl)phenylamino)-1,3,4,6,7,9,9b-heptaazaphenalene;
2,5,8-tris-(3-nitrophenylamino)-1,3,4,6,7,9,9b-heptaazaphenalene;
2,5,8-tris-(4-(2-butyloctyloxycarbonyl)phenylamino)-1,3,4,6,7,9,9b-heptaazaphenalene;
2,5,8-tris-(4-(2-hexyldecyloxycarbonyl)phenylamino)-1,3,4,6,7,9,9b-heptaazaphenalene;
2,5,8-tris-(4-(dodecyloxycarbonyl)phenylamino)-1,3,4,6,7,9,9b-heptaazaphenalene;
2,5,8-tris-(4-(hexyldecyloxycarbonyl)phenylamino)-1,3,4,6,7,9,9b-heptaazaphenalene;
2,5,8-tris-(4-(octyldecyloxycarbonyl)phenylamino)-1,3,4,6,7,9,9b-heptaazaphenalene;
2,5,8-tris-((3-(E)-styrylphenyl)amino)-1,3,4,6,7,9,9b-heptaazaphenalene;
2,5,8-tris-(4-((3,5,5-trimethylhexyloxy)carbonylphenylamino)-1,3,4,6,7,9,9b-heptaazaphenalene;
2,5-bis-(3-(methoxy)phenylamino)-8-(2-(1-methyl-1H-pyrrol-2-yl)-1,3,4,6,7,9,9b-heptaazaphenalene;
2,5,8-tris-(4-((2-ethylhexyl)carbamoyl)phenylamino)-1,3,4,6,7,9,9b-heptaazaphenalene;
2,5,8-tris-(4-(dodecyloxycarbonyl)phenylamino)-1,3,4,6,7,9,9b-heptaazaphenalene;
2,5,8-tris-(4-(1,3,3-trimethylbicyclo[2.2.1]heptan-2-yloxy)phenylamino)-1,3,4,6,7,9,9b-heptaazaphenalene;
2,5,8-tris-(1H-indol-5-ylamino)-1,3,4,6,7,9,9b-heptaazaphenalene;
2,5,8-tris-(4-((3,7-dimethyloctyloxy)carbonyl)phenylamino)-1,3,4,6,7,9,9b-heptaazaphenalene;
2,5,8-tris-(2-amino-4,5-dimethylphenylamino)-1,3,4,6,7,9,9b-heptaazaphenalene.

20. (canceled)

21. (canceled)

22. (canceled)

23. (canceled)

24. (canceled)

25. (canceled)

26. (canceled)

27. A method for obtaining a heptaazaphenalene derivative of general formula (I) according to claim 1 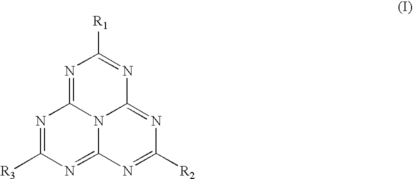
the method being selected from one of the following:
(a) having R₁, R₂ and R₃ be the same and represent —NR₅R₆, and having R₅ and R₆ be as defined in claim 1, and reacting the 2,5,8-trichloro-1,3,4,6,7,9,9b-heptaazaphenalene derivative of formula (IV) with a derivative of general formula (V) 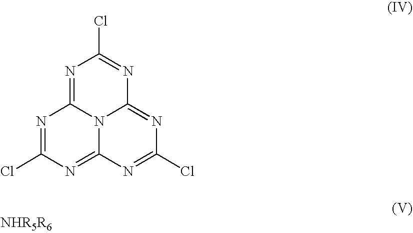
in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, between room temperature and the boiling temperature of the solvent, or between 50° C. and the boiling temperature of the solvent, optionally in the presence of an organic base comprising diisopropylethylamine, triethylamine or pyridine, or an inorganic base comprising potassium carbonate, sodium hydroxide, sodium carbonate, cesium carbonate or sodium bicarbonate;
(b) having R₁, R₂ and R₃ be the same and represent —NR₅R₆; having R₅ and R₆ as defined in claim 1; and having one of the radicals R₁, R₂ and R₃ be different from the other two, and (i) making the 2,5,8-trichloro-1,3,4,6,7,9,9b-heptaazaphenalene derivative of formula (IV) react with a derivative of general formula (V) where R₅ and R₆ are as defined in claim 1, in a solvent comprising 1,4-dioxane, tetrahydrofuran, toluene, xylene (mixture of isomers), N,N-dimethylformamide, N-methylpyrrolidone or acetone, at a temperature that ranges between 0° C. and the boiling temperature of the solvent, between room temperature and the boiling temperature of the solvent, or between 50° C. and the boiling temperature of the solvent; optionally in the presence of an organic base comprising diisopropylethylamine, triethylamine or pyridine, or an inorganic base comprising potassium carbonate, sodium hydroxide, sodium carbonate, cesium carbonate or sodium bicarbonate; and (ii) adding to the mixture resulting from the preceding stage a second derivative of general formula (V) different from the one used in stage (i) and submitting to reflux;
(c) having R₁, R₂ and R₃ be different from each other and represent —NR₅R₆, having R₅ and R₆ be as defined in claim 1, (i) making the 2,5,8-trichloro-1,3,4,6,7,9,9b-heptaazaphenalene derivative of formula (IV) react with a derivative of general formula (V), where R₅ and R₆ are as defined in claim 1, in a solvent comprising 1,4-dioxane, tetrahydrofuran, toluene, tetrahydrofuran, xylene (mixture of isomers), N,N-dimethylformamide, N-methylpyrrolidone or acetone, at a temperature that ranges between 0° C. and the boiling temperature of the solvent, between room temperature and the boiling temperature of the solvent, or between 50° C. and the boiling temperature of the solvent; optionally in the presence of an organic base comprising diisopropylethylamine, triethylamine or pyridine, or an inorganic base comprising potassium carbonate, sodium hydroxide, sodium carbonate, cesium carbonate or sodium bicarbonate; (ii) adding to the resulting mixture a derivative of general formula (V) different from the one used in the preceding stage where one of R₅ and R₆ are as defined in claim 1, and submitting to reflux; and (iii) adding to the mixture resulting from stage (ii) a derivative of general formula (V) different from the one used in stages (i) and (ii) where one of R₅ and R₆ are as defined in claim 1;
(d) having one of the radicals R₁, R₂ and R₃ represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and having the other two radicals be the same and represent —NR₅R₆, where R₅ and R₆ are as defined in claim 1, and reacting of a derivative of general formula (VII) with a derivative of general formula (V): 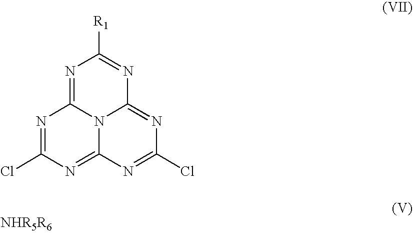
wherein R₁ is an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; and having R₅ and R₆ be as defined in claim 1, in an inert solvent comprising 1,4-dioxane, tetrahydrofuran, toluene, xylene (mixture of isomers) N,N-dimethylformamide, N-methylpyrrolidone or acetone, at a temperature that ranges between 0° C. and the boiling temperature of the solvent, between room temperature and the boiling temperature of the solvent or between 50° C. and the boiling temperature of the solvent.

28. (canceled)

29. (canceled)

30. (canceled)

31. (canceled)

32. (canceled)

33. (canceled)

35. (canceled)

36. A heptaazaphenalene derivative according to claim 1 for use as a UV radiation-absorbing agent.

37. A dermatological, cosmetic, pharmaceutical or veterinary formulation comprising a compound according to general formula (I) 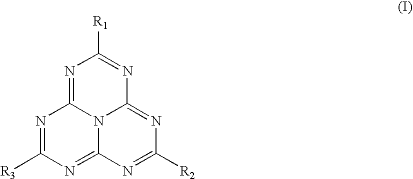
wherein
R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;
R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;
R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S;
and at least one dermatologically, cosmetically, and/or pharmaceutically acceptable carrier or excipient.

38. (canceled)

39. (canceled)

40. (canceled)

41. (canceled)

42. (canceled)

43. (canceled)

44. (canceled)

45. A medicament comprising a compound according to general formula (I): 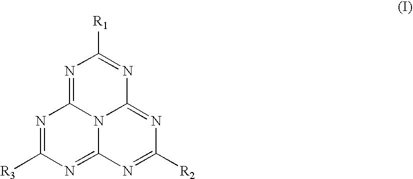
wherein
R₁, R₂ and R₃ are identical or different from each other and represent an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; an —OR₄ radical; or an —NR₅R₆ radical;
R₄ represents hydrogen, an optionally substituted saturated or unsaturated linear or branched alkyl radical that contains from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 14 atoms that can contain 1, 2 or 3 heteroatoms selected from O, N and S;
R₅ and R₆ are identical or different from each other and represent hydrogen; an optionally substituted, saturated or unsaturated, linear or branched alkyl radical having from 1 to 18 carbon atoms; an optionally substituted C₃-C₁₂ cycloalkyl radical; an optionally substituted mono- or polycyclic aryl radical; an optionally substituted saturated, unsaturated or aromatic heterocyclic radical having from 5 to 10 atoms that can contain 1, 2 or 3 heteroatoms selected from O, N and S; or R₅ and R₆ are fused to form together with the nitrogen a saturated, unsaturated or aromatic mono- or polycyclic ring system having from 4 to 10 atoms that can optionally contain 1 or 2 heteroatoms selected from N, O and S;
and at least one pharmaceutically acceptable carrier or excipient.

46. (canceled)

47. A formulation according to any of claim 37, which further includes at least one organic, micronized organic or inorganic filter against solar radiation, or at least one active substance.

48. (canceled)

49. A method of use of a heptaazaphenalene derivative or mixture of heptaazaphenalene derivatives according to claim 1 as a UV radiation filtering agent in a cosmetic, dermatological, veterinary or pharmaceutical composition, the method comprising administering to a subject a cosmetically, dermatologically and/or pharmaceutically effective amount of a heptaazaphenalene derivative according to claim 1 together with cosmetically and/or dermatologically acceptable carriers or excipients.

50. A method of use of a heptaazaphenalene derivative or mixture of heptaazaphenalene derivatives according to claim 1 for the protection, prophylaxis and/or coadjuvant treatment of on healthy or diseased skin, lips and/or related tissues of a mammal against ultraviolet radiation, the method comprising administering to a subject a cosmetically, dermatologically and/or pharmaceutically effective amount of a heptaazaphenalene derivative according to claim 1 together with cosmetically, dermatologically and/or pharmaceutically acceptable carriers or excipients.

51. (canceled)

52. (canceled)

53. A method of use of a derivative according to claim 1 as a photostabiliser of polymers, or as an ultraviolet radiation-filtering agent in textile fibres, the method comprising applying to a polymer or a textile fibre and effective amount of the heptaazaphenalene derivative according to claim 1.

54. (canceled)
